(19) United States
(12) Patent Application Publication
(10) Pub. No.: US 2024/0158814 A1
(43) Pub. Date: May 16, 2024

(54) DENDRITIC PEPTIDE CONJUGATED POLYMERS FOR EFFICIENT INTRACELLULAR DELIVERY OF NUCLEIC ACIDS TO IMMUNE CELLS

(71) Applicant: Northwestern University, Evanston, IL (US)

(72) Inventors: Evan Alexander Scott, Evanston, IL (US); Sijia Yi, Evanston, IL (US)

(21) Appl. No.: 18/551,095

(22) PCT Filed: Mar. 17, 2022
(86) PCT No.: PCT/US22/20827

§ 371 Date: Sep. 18, 2023

Related U.S. Application Data
(60) Provisional application No. 63/162,507, filed on Mar. 17, 2021.

(51) Int. Cl. C12N 15/88; A61K 47/64; A61K 47/69; C12N 5/00; C12N 5/0784

(52) U.S. Cl. CPC C12N 15/88; A61K 47/6455; A61K 47/6935; C12N 5/0018; C12N 5/0639

(57) ABSTRACT

The present invention provides nanocarriers for delivering polynucleotide sequences to cells, specifically immune cells, including dendritic cells and methods of use. The methods provide improved delivery and reduced toxicity over prior methods. The method of the present disclosure provide a system for delivering nucleic acids to a cell, consisting of a synthetic PEG-b-PPS-linker-DP polymer for producing nanostructures comprising a poly(ethylene glycol)-blockpoly (propylene sulfide) copolymer (PEG-b-PPS) conjugated with a dendritic-specific branched cationic peptide (DP). The system provides a non-toxic in-vitro method of delivering a polynucleotide to immune cells, including dendritic cells, comprising of contacting the cell in cell culture medium with a nanocarrier wherein the method is non-toxic to the cells. The methods described in the invention can be used for treating a subject in need of gene therapy, comprising administering to the subject an effective amount of the system comprising of a polynucleotide, wherein the polynucleotide contains a gen of interest for gene therapy.

### CROSS-REFERENCE TO RELATED APPLICATIONS

This application claims priority to U.S. Provisional Application No. 63/162,507 filed on Mar. 17, 2021, the contents of which are incorporated by reference in its entirety.

### STATEMENT REGARDING FEDERALLY SPONSORED RESEARCH

This invention was made with government support under HL132390 awarded by the National Institutes of Health and under 1453576 awarded by the National Science Foundation. The government has certain rights in the invention.

### BACKGROUND OF THE INVENTION

Recent breakthroughs in genetic engineering offer an unparalleled opportunity for cancer immunotherapy, antiviral therapies, and DNA vaccines in the treatment of cancer and infectious diseases[1-4]. Notwithstanding a large number of ongoing trials on gene editing, successful gene therapy products are very limited[5]. This mainly ascribed to challenges in the effective introduction of nucleic acids into target cells. An efficient DNA delivery system must overcome several obstacles such as protection from enzyme degradation, cellular internalization, escape from endo-lysosomes, and cytosolic cargo release.

As natural gene delivery vehicles, viruses exhibit unprecedented performance for gene expression; however, the safety concerns, such as insertional mutagenesis and their intrinsic immunogenicity hinder their use in clinical settings[6]. More importantly, the limited DNA-carrying capacity (<8 kb) is another significant restriction for viral-based vectors[7]. To overcome the obstacles of viral counterparts, non-viral vectors and synthetic carriers, including lipids, polymers, peptides, and inorganic nanomaterials have attracted increasing attention because of their limited immunogenicity, flexible packaging capacity, and relatively easy to synthesize and to manufacture [8]. However, existing materials still face challenges such as non-efficient endosomal escape, substantial toxicity, and low gene transfection/cellular expression, especially in some of the most exciting target cell types, such as immune cells. For example, cationic lipids, which are considered the most widely used non-viral vectors for nucleic acid delivery in most immortalized cell lines, but many blood and immune cells remain recalcitrant [9]. Thus, systems for delivering nucleic acids to some hard-to-transfected cells, such as primary cells and immune cells, are in high demand.

Dendritic or branched cationic peptides have a three-dimensional (3D) architecture with multiple functional groups, making them efficient gene delivery materials for highly negatively charged nucleic acids [10]. It has been reported that dendritic structures can significantly enhance the interaction of peptides with DNA, considerably improve the cargo packaging, and increase the transfection efficiency in diverse cell types, compared to linear structures [11]. Notably, a variety of parameters would affect the activity of dendritic peptides, including generations based on the layer of peptide branching, molecular weight, functional or branching units, and charge distribution. For example, three-generated peptide dendrimers with lower molecular weight to charge ratios and charges distributed over the whole dendritic structure were demonstrated to be the best transfection reagents for DNA delivery [12]. Despite their obvious potential, cationic peptide dendrimers were complexed with nucleic acid mainly through electrostatic interactions, which are generally unstable in serum and with potential cytotoxicity, limiting their applications in gene therapy.

There is a need for a nucleic acid delivery system that overcomes the aforementioned issues with the present available delivery systems.

### SUMMARY OF THE INVENTION

The present disclosure provides compositions and methods for delivering nucleic acids to cells. In one aspect, the present disclosure provides a synthetic PEG-b-PPS-linker-DP polymer for producing nanostructures comprising a poly(ethylene glycol)-block-poly(propylene sulfide) copolymer (PEG-b-PPS) conjugated with a dendritic-specific branched cationic peptide (DP). In some aspects, the linker is a disulfide bond (ss). In some aspects, the polymer comprises PEGₘ-b-PPSₙ, wherein m and n are each integers selected from 1-500.

In another aspect, the disclosure provides a system for delivering nucleic acids to a cell, the system comprising: (a) a nanostructure comprising poly(ethylene glycol)-block-poly(propylene sulfide) copolymer (PEG-b-PPS) conjugated with a dendritic-specific branched cationic peptide (DP) via a linker (PEG-b-PPS-linker-DP); and (b) a polynucleotide selected from the group consisting of DNA and RNA. In some aspects, the linker is a disulfide bond (e.g., PEG-b-PPS-ss-DP). In some aspects, the delivery is achieved in vitro. In particular aspects, the method is for in vitro delivery to immune cells, including dendritic cells.

In another aspect, the present disclosure provides a method of delivering a polynucleotide sequence to a cell, the method comprising contacting the polynucleotide via the nanostructures described herein to a cell described herein in order for the cell to incorporate the polynucleotide into the cell.

In another aspect, the disclosure provides a non-toxic in vitro method of delivering a polynucleotide to a cell, the method comprising (a) contacting the cell in cell culture medium with a nanocarrier comprising (i) PEGₘ-b-PPSₙ conjugated to a DP peptide, wherein m and n are integers from 1-500, and (ii) a polynucleotide, and (b) culturing the cells for a sufficient time for delivery of the polynucleotide to the cell nucleus wherein the method is non-toxic to the cells.

In some aspects, the method is for in vitro delivery. In particular aspects, the method is for in vitro delivery to immune cells, including dendritic cells.

Further aspects include a cell system comprising the polynucleotide.

In a further embodiment, the disclosure provides a method of transfecting an immune cell to deliver a polynucleotide sequence to the nucleus of the immune cell, the method comprising contacting the immune cell with the nanocarrier system described herein for a sufficient time to deliver the polynucleotide sequence to the nucleus of the immune cell. In some aspects the method is in vitro.

In yet another embodiment, the disclosure provides a method of treating a subject in need of gene therapy, the method comprising administering to the subject an effective amount of the system described herein, wherein the system comprises a polynucleotide comprising a gene of interest for gene therapy.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1. Schematic illustration showing the design and structure of PEGₘ-b-PPSₙ-ss-DP (PPDP) gene delivery system, and the strategy for enhancing intracellular delivery of DNA.

FIG. 2. Cytotoxicity of DNA-PPDP nanocomplexes in vitro. Cell viability of DNA-PPDP nanocomplexes was determined using MTT assays in RAW264.7 cells at different polymer to DNA ratio (15:1, 30:1, 60:1, 120:1). Untreated cells (Control), naked DNA (DNA), DNA-Lipofectamine 2 k complexes (Lipo2K), DAN-PEI complexes (PEI with the molecular weight of 25 kDa, PEI to DNA weight ratio of 5:1 and 10:1) were included as control groups.

FIG. 3. Transfection of small sized plasmid DNA with PPDP nanovectors in different immune cells. A, Schematic of small sized plasmid DNA (pCMV-EGFP, 4.6 Kb). The pCMV-EGFP contains a CMV promoter, Neo/Kan resistant genes and an EGFP reporter gene. B, Transfection efficiency of DNA by a variety of PPDP nanovectors was evaluated with the percentage of fluorescence-expression cells on RAW264.7 macrophages. DNA-PPDP nanocomplexes were prepared at a weight ratio of 60:1 (PPDP:DNA). DNA-DP1 complexes were prepared at a weight ratio of 20:1 (DP1:DNA). Transfections by Lipo2K was performed according to the manufacture's instruction. Transfection efficiency of DNA-PPDP2 (C) and DNA-PPDP5 (D) at different PPDP to DNA weight ratio (15:1, 30:1, 60:1, 120:1) with the same amount of DNA on RAW264.7 macrophages. E-F, Flow cytometry analysis of bone-marrow derived dendritic cells (BMDCs) transfected with pCMV-EGFP (4.6 Kb) using PPDP2 and PPDP5 nanovectors with the PPDP to DNA weight ratio of 60:1. Naked DNA and DNA-Lipo2K complexes (Lipo2K) were introduced as negative and positive control groups. The same amount of DNA was used for all transfection experiments. All statistical data are presented as means±s.d. N=3 for each group. Two-tailed t-tests were used for statistical analysis: **p<0.01, ***p<0.001.

FIG. 4. Transfection of large sized plasmid DNA with PPDP nanovectors in a variety of cells. A, Schematic of large sized plasmid DNA (pEFS-RFP, 11.7 Kb). The pCMV-EGFP contains a EFS promoter, a NSL gene, a Cas9 expression gene, a AMP resistant gene and a RFP reporter gene. B, Transfection efficiency of pEFS-RFP (11.7 Kb) plasmid DNA by a variety of PPDP nanovectors was evaluated with the percentage of RFP-positive cells on RAW264.7 macrophages. DNA-PPDP nanocomplexes were prepared at a weight ratio of 60:1 (PPDP:DNA). Transfections by Lipo2K was performed according to the manufacture's instruction. C, Representative confocal image of PPDP2-mediated delivery of pEFS-RFP (11.7 Kb) plasmid DNA into RAW264.7 macrophages. Transfection efficiency of DNA-PPDP2 (D) and DNA-PPDP5 (E) at different PPDP to DNA weight ratio (10:1, 20:1, 40:1, 60:1, 100:1) with the same amount of DNA on RAW264.7 macrophages. F, Transfection efficiency of pEFS-RFP (11.7 Kb) plasmid DNA delivered with PPDP2 and PPDP5 at a weight ratio of 60:1 (PPDP:DNA) on NIH3T3 fibroblasts. G, Representative confocal image of PPDP2-mediated delivery of pEFS-RFP (11.7 Kb) plasmid DNA into NIH3T3 fibroblasts. H-I, Flow cytometry analysis of bone-marrow derived dendritic cells (BMDCs) transfected with pEFS-RFP (11.7 Kb) using PPDP2 and PPDP5 nanovectors with the PPDP to DNA weight ratio of 60:1. Naked DNA and DNA-Lipo2K complexes (Lipo2K) were introduced as negative and positive control groups. The same amount of DNA was used for all transfection experiments. Scale bar=10 μm. All statistical data are presented as means±s.d. N=3 for each group. Two-tailed t-tests were used for statistical analysis: **p<0.01, ***p<0.001, ****p<0.0001.

FIG. 5. Endosomal escape and cytosolic delivery of PPDP2/pDNA complexes in RAW 264.7 macrophages. RAW 264.7 macrophages were incubated with PPDP2/AF488-labeled S-pDNA (pcDNA3.1, 5.4 kb) nanocomplexes formed using a PPDP2 to pDNA weight ratio of 60:1. Representative confocal images display PPDP2/S-pDNA complexes within cells after 1 h, 4 h, and 18 h incubation periods. LysoTracker red was used to label late endosomes/lysosomes. Nuclei were stained with DAPI (blue). Co-localization of green plasmid DNA and red endo/lysosomes appears as yellow in the images. Scale bar=10 μm.

FIG. 6. Schematic representation of PEGₘ-b-PPSₙ-ss-Dendritic Peptide (PPDP) polymer synthesis.

FIG. 7. Cytotoxicity of DNA-PPDP nanocomplexes in vitro. Cell viability of DNA-PPDP nanocomplexes was determined using MTT assays in RAW264.7 cells at different polymer to DNA ratio (15:1, 30:1, 60:1, 120:1).

FIG. 8. (A) RAW264.7 macrophages were transfected with pCMV-EGFP (4.6 Kb) using a variety of PPDP nanovectors. Mean fluorescent intensity (MFI) was determined by flow cytometry. DNA-PPDP nanocomplexes were prepared at a weight ratio of 60:1 (PPDP:DNA). DNA-DP1 complexes were prepared at a weight ratio of 20:1 (DP1:DNA). Transfections by Lipo2K was performed according to the manufacture's instruction. The MFI of RAW264.7 macrophages transfected with DNA-PPDP2 (B) and DNA-PPDP5 (C) at different PPDP to DNA weight ratio (15:1, 30:1, 60:1, 120:1) with the same amount of DNA. All statistical data are presented as means±s.d. N=3 for each group. Two-tailed t-tests were used for statistical analysis: **p<0.01, ***p<0.001.

FIG. 9. The MFI of bone-marrow dendritic cells (BMDCs) transfected with DNA-PPDP2 and DNA-PPDP5 at the PPDP to DNA weight ratio of 60:1. Lipo2K was used as a positive control to deliver pCMV-EGFP (4.6 Kb). All statistical data are presented as means s.d. N=3 for each group. Two-tailed t-tests were used for statistical analysis: *p<0.05.

FIG. 10. (A) RAW264.7 macrophages were transfected with pEFS-RFP (11.7 Kb) using a variety of PPDP nanovectors. Mean fluorescent intensity (MFI) was determined by flow cytometry. DNA-PPDP nanocomplexes were prepared at a weight ratio of 60:1 (PPDP:DNA). DNA-DP1 complexes were prepared at a weight ratio of 20:1 (DP1:DNA). Transfections by Lipo2K was performed according to the manufacture's instruction. The MFI of RAW264.7 macrophages transfected with DNA-PPDP2 (B) and DNA-PPDP5 (C) at different PPDP to DNA weight ratio (10:1, 20:1, 40:1, 60:1, 100:1) with the same amount of DNA. All statistical data are presented as means±s.d. N=3 for each group. Two-tailed t-tests were used for statistical analysis: **p<0.01, ***p<0.001, ****p<0.0001.

FIG. 11. Transfection of PPDP/L-pDNA nanovector in fibroblasts, dendritic cells, and T cells. NIH 3T3, BMDC, and Jurkat T cells were transfected with L-pDNA (pL-CRISPR.EFS.tRFP, 11.7 kb) using PPDP2 and PPDP5 nanovectors with the PPDP to pDNA weight ratio of 60:1. a) Representative confocal image of PPDP2/L-pDNA complexes demonstrated the transfection of L-pDNA after cellular uptake. Scale bar=20 μm. b) Percentage of transfection efficiency in NIH 3T3 cells. c) Flow cytometry histogram and d) the percentage of transfected cells in BMDCs. e) Flow cytometry histogram and f) the percentage of transfected Jurkat T cells. Naked pDNA and Lipo2K/pDNA complexes (Lipo2K) were included as negative and positive controls, respectively. Data are presented as the mean±SD (n=3-4). Significance was determined by ANOVA with post hoc Tukey's multiple comparisons test (5% significance level). ***p<0.001, ****p<0.001. Scale bar=10 μm.

FIG. 12. The MFI of NIH3T3 fibroblasts (A) and bone-marrow dendritic cells (BMDCs) (B) transfected with DNA-PPDP2 and DNA-PPDP5 at the PPDP to DNA weight ratio of 60:1. Lipo2K was used as a positive control to deliver pEFS-RFP (11.7 Kb). All statistical data are presented as means±s.d. N=3 for each group. Two-tailed t-tests were used for statistical analysis: **p<0.01, ***p<0.001, ****p<0.0001.

FIG. 13. ¹H NMR spectra of PEGₙ-b-PPSₘ-pds. ¹H NMR (400 MHz. Chloroform-d); a) PPDP2, b) PPDP3, c) PPDP4, d) PPDP5, e) PPDP6, and f) PPDP7. δ: 3.6 (s, 68H, PEG), 3.3 (s, 3H, PEG-OCH₃), 2.9 (m, 2H/unit, —S—CH₂CH(CH₃)—S—), 2.6 (m, 1H/unit, —S—CH₂CH(CH₃)—S—), 1.3 (m, 3H/unit, —S—CH₂CH(CH₃)—S—).

FIG. 14. High tension (HT) voltage during CD spectra acquisition. The HT voltage during the collection of spectra for a) PPDP2, and b) peptide control samples is displayed.

FIG. 15. Characterization of PPDP/pDNA nanovector. Agarose gel electrophoresis retardation of PPDP/pDNA nanocomplexes with the different polymer to pDNA ratio from 1:1 to 60:1. Naked pDNA, Lipofectamine 2000/pDNA complexes (Lipo2K), PEI/pDNA complexes (PEI with the molecular weight of 25 kDa), and Dendritic peptide (DP1)/pDNA complexes were included as control groups. Optimizing the formulation of PPDP with pDNA. a) PPDP/S-pDNA complexes, b) PPDP/L-pDNA complexes. (triangle: The well-encapsulated pDNA in PPDP/pDNA nanocomplex). Effect of the mass ratio of PPDP2 and 5 to c) S-pDNA and, d) L-pDNA on the particle size (red) and zeta potential (blue) of complexes. (asterisk: a ratio of formulation optimization). Representative image of PPDP/pDNA nanocomplexes from cryo-transmission electron microscopy. e) PPDP/S-pDNA complexes and f) PPDP/L-pDNA complexes at weight ratio of 60:1 (PPDP:pDNA). Scale bars (black)=100 nm. Data are presented as the mean±SD (n=3).

FIG. 16. Characterization of PEGₘ-b-PPSₙ-ss-DP (PPDP). a) Schematic illustration of PEGₘ-b-PPSₙ-ss-DP (PPDP) Polymer. b) Representative cryo-transmission electron microscopy (cryo-TEM) image and c) size distribution of PPDP2. Scale bars=100 nm. d) FT-IR spectra of PEG-b-PPS (green), dendritic peptide (black), and PPDP2 (red). The regions highlighted in blue correspond to the amide I (1700-1600 cm⁻¹) and amide II (1590-1520 cm⁻¹) bands. e) PPDP2 spectra with PEG-b-PPS contributions subtracted. Circular dichroism (CD) spectroscopy analysis of the helical structure of the DP peptide f) in assembled nanostructures or g) in free form.

FIG. 17. Gel retardation assay of PPDP/pDNA nanovector. Agarose gel electrophoresis retardation of PPDP/pDNA nanocomplexes with a different polymer to pDNA ratio from 1:1 to 120:1. Dendritic peptide (DP1)/pDNA complexes and PEI/pDNA complexes (PEI with the molecular weight of 25 kDa) were included as control groups. The well-encapsulated pDNA in PPDP/pDNA nanocomplex are indicated by a red triangle. a) PPDP/S-pDNA complexes, b) PPDP/L-pDNA complexes.

FIG. 18. Cytotoxicity of PPDP/pDNA nanovector in RAW 264.7 cells. The murine macrophage cell line RAW 264.7 cells were incubated PPDP/pDNA nanocomplexes with a different polymer to pDNA ratio (30:1, 60:1, 120:1) in a 96-well plate at a cell density of 3×10⁴/well for 24 h at 37° C. Untreated cell (Control), Lipofectamine 2000/pDNA complexes (Lipo2K), PEI/pDNA complexes (PEI with the molecular weight of 25 kDa), and Dendritic peptide (DP1)/pDNA complexes were included as control groups. The cell viability was then measured by the MTT assay. a) PPDP/S-pDNA complexes, b) PPDP/L-pDNA complexes. (asterisk: ratio of formulation optimization). Data are presented as the mean±SD (n=3).

FIG. 19. Transfection of PPDP/L-pDNA nanovector in a variety of cell lines. NIH 3T3, BMDMs, and Jurkat were transfected with L-pDNA (pL-CRISPR.EFS.tRFP,11.7 Kb) using PPDP2 and PPDP5 nanovectors with the PPDP to pDNA weight ratio of 60:1. The mean fluorescence intensity (MFI) of transfection efficiency in a) NIH3T3, b) BMDC, and c) Jurkat cells. Naked pDNA and Lipo2K/pDNA complexes (Lipo2K) were introduced as negative and positive control groups. The transfection efficiency was analyzed by flow cytometry. Data are presented as the mean±SD (n=3). Two-tailed t-tests were used for statistical analysis: **p<0.01, ***p<0.001.

FIG. 20. Transfection of PPDP/L-pDNA nanovector in a variety of cell lines. NIH 3T3, BMDC, and Jurkat cells were transfected with L-pDNA (pL-CRISPR.EFS.tRFP,11.7 Kb) using PPDP2 and PPDP5 nanovectors with the PPDP to pDNA weight ratio of 60:1. a) Representative confocal image of PPDP2/L-pDNA complexes demonstrated the transfection of L-pDNA after cellular uptake. Scale bar (white)=20 μm. b) Percentage of transfection efficiency in NIH3T3. c) Histogram and d) percentage of transfection efficiency in BMDCs. e) Histogram and f) percentage of transfection efficiency in Jurkat cells. Naked pDNA and Lipo2K/pDNA complexes (Lipo2K) were introduced as negative and positive control groups. The transfection efficiency was analyzed by flow cytometry. Data are presented as the mean±SD (n=3-4). Two-tailed t-tests were used for statistical analysis: *p<0.05, **p<0.01, ***p<0.001.

FIG. 21. Transfection of PPDP/pDNA nanovector in RAW 264.7 cells. The murine macrophage cell line RAW 264.7 cells were incubated with indicated materials for 48 h. a) Schematic of small size plasmid DNA (pCMV-DsRed, 4.6 Kb). The pCMV-DsRed contains a CMV promoter, Neo/Kan resistant genes, and a DsRed reporter gene. b) Transfection efficiency of S-pDNA by a variety of PPDP nanovectors was evaluated with the percentage of fluorescence-expression cells on RAW 264.7 cells. PPDP/S-pDNA nanocomplexes were prepared at a weight ratio of 60:1 (PPDP:pDNA). Transfections by Lipo2K were performed according to the manufacture's instruction. Transfection efficiency of c) PPDP2/S-pDNA and PPDP5/S-pDNA complexes at different PPDP to pDNA weight ratio (15:1, 30:1, 60:1, 120:1). d) Schematic of large sized plasmid DNA (pL-CRISPR.EFS.tRFP, 11.7 Kb). The pL-CRISPR.EFS.tRFP contains a EFS promoter, a NSL gene, a Cas9 expression gene, a AMP resistant gene, and a RFP reporter gene. e) Transfection efficiency of L-pDNA by a variety of PPDP nanovectors was evaluated with the percentage of fluorescence-expression cells on RAW 264.7 cells Transfection efficiency of f) PPDP2/L-pDNA and PPDP5/L-pDNA complexes at different PPDP to pDNA weight ratio (10:1, 40:1, 60:1, 100:1). g) Representative confocal image of PPDP2/L-pDNA complexes (with the PPDP2 to pDNA weight ratio of 60:1) demonstrated the transfection of L-pDNA after cellular uptake. Scale bar (white)=20 μm. The transfection efficiency analyzed by flow cytometry. Data are presented as the mean±SD (n=3). Two-tailed t-tests were used for statistical analysis: *p<0.05, **p<0.01, ***p<0.001.

FIG. 22. Transfection of PPDP/pDNA nanovector in RAW 264.7 cells. The murine macrophage cell line RAW 264.7 cells were incubated with indicated materials for 48 h. a) Transfection efficiency of S-pDNA by a variety of PPDP nanovectors was evaluated with the mean fluorescence intensity (MFI) on RAW 264.7 cells. PPDP/S-pDNA nanocomplexes were prepared at a weight ratio of 60:1 (PPDP:pDNA). Transfections by Lipo2K were performed according to the manufacture's instruction. Transfection efficiency of b) PPDP2/S-pDNA and PPDP5/S-pDNA complexes at different PPDP to pDNA weight ratio (15:1, 30:1, 60:1, 120:1). c) Transfection efficiency of L-pDNA by a variety of PPDP nanovectors was evaluated with the mean fluorescence intensity (MFI) on RAW 264.7 cells. Transfection efficiency of d) PPDP2/L-pDNA and PPDP 5/L-pDNA complexes at different PPDP to pDNA weight ratio (10:1, 40:1, 60:1, 100:1). The transfection efficiency analyzed by flow cytometry. Data are presented as the mean±SD (n=3). Two-tailed t-tests were used for statistical analysis: *p<0.05, **p<0.01, ***p<0.001.

FIG. 23. Cryo-TEM images of PPDP/pDNA nanovector images. (a) are images of PPD2 and (b) are PPDP5. Scale bars (white)=100 nm.

FIG. 24. PPDP2 and PPDP5 achieve higher plasmid DNA transfection efficiencies and reporter expression levels than Lipo2K in a macrophage-based screen. RAW 264.7 macrophages were incubated with the specified materials for 48 h. a) Schematic of the model small size plasmid DNA (pCMV-DsRed, 4.6 kb), containing a CMV promoter and a DsRed reporter gene. b) Transfection efficiency of S-pDNA using PPDP nanovectors (PPDP2-PPDP7) quantified as the percentage of cells expressing fluorescent reporter proteins. c) Transfection efficiency of S-pDNA plotted with the mean fluorescence intensity (MFI) of cells expressing the DsRed reporter. d) Schematic of the model large size plasmid DNA (pL-CRISPR.EFS.tRFP, 11.7 kb) used in this study, containing an EFS promoter with NSL, Cas9, and RFP reporter genes. e) Transfection efficiency of L-pDNA by PPDP nanovectors. f) Transfection efficiency and MFI of cells expressing the RFP reporter. g) Fold difference in the PPDP-mediated plasmid transfection efficiency (left) and reporter expression level (right) compared to Lipo2K. The rank of PPDP2 and PPDP5 is annotated below each heat map. In all cases, a 60:1 PPDP:pDNA weight ratio was used. Data are presented as the mean±SD (n=3). Transfections using were performed per manufacturer instructions. For panels (b, e), significant differences were determined by ANOVA with post hoc Tukey's multiple comparisons test (5% significance level). *p<0.05, **p<0.005, ***p<0.001, ****p<0.0001. Comparisons to control and pDNA (gray bars), Lipo2k (red bars), and within PPDP treatment groups (black bars) are presented in the specified colors.

FIG. 25. Cytotoxicity of PPDP/pDNA nanovector in RAW 264.7 macrophages. RAW 264.7 macrophages were incubated with PPDP/pDNA nanocomplexes that were prepared with a different polymer to pDNA ratio (30:1, 60:1, 120:1) for 24 h at 37° C. Untreated cells (Control), Lipo2K/pDNA complexes (Lipo2K), PEI/pDNA complexes (PEI with the molecular weight of 25 kDa), and dendritic peptide (DP)/pDNA complexes were included as benchmarks. The cell viability for a) PPDP/S-pDNA complexes and b) PPDP/L-pDNA complexes was then measured by the MTT assay. The asterisk indicates the experimentally determined optimal ratio. Data are presented as the mean±SD (n=3).

FIG. 26. The 60:1 PPDP:p-DNA ratio was optimal for transfection of macrophages with both small and large plasmids. The transfection efficiency in RAW 264.7 macrophages was determined by flow cytometry using PPDP2 (purple bar plots) or PPDP5 (red bar plots) complexed with a) the small model plasmid (S-pDNA; pCMV-DsRed, 4.6 kb), or b) the large model plasmid (L-pDNA; pL-CRISPR.EFS.tRFP, 11.7 kb) at the specified PPDP:pDNA ratios. Data are presented as the mean±SD (n=3). Significant differences were determined by ANOVA with post hoc Tukey's multiple comparisons test (5% significance level). *p<0.05, **p<0.005, ***p<0.001, ****p<0.0001. c) Representative confocal image of reporter gene expression by macrophages transfected with PPDP2/L-pDNA (60:1) or Lipo2K/L-pDNA. Cellular background (control) and cells transfected with naked L-pDNA are also presented. Scale bar=20 μm.

FIG. 27. Gel permeation chromatography (GPC) before and after conjugation of PEG-b-PPS to the dendritic peptide (DP) to form PPDP. Analysis was performed with 10 mM lithium bromide in N,N-Dimethylformamide as the mobile phase. GPC was conducted using PLgel columns with refractive index and UV-Vis detectors. a) PEG17-b-PPS80-pds b) PEG17-b-PPS80-ss-DP.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

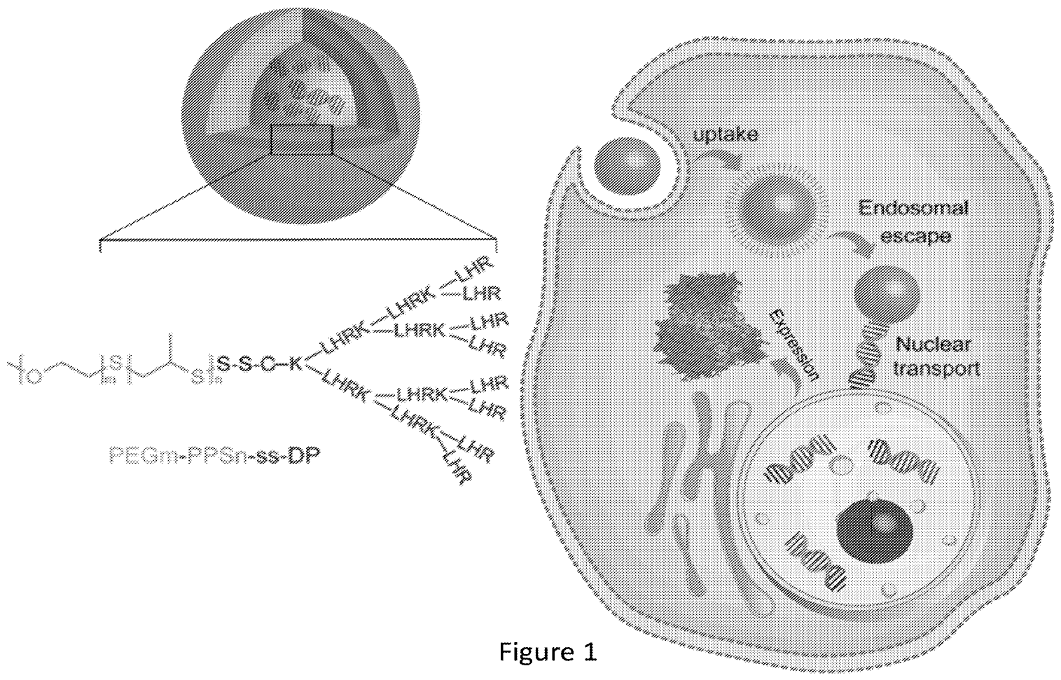

Disclosed herein is a novel gene delivery system based on polymer nanocarriers engineered for efficient cytosolic delivery of polynucleotides (FIG. 1). PEG-b-PPS polymers conjugated with a dendritic peptide (DP) (PPDP) are assembled into stable nanostructures that encapsulate nucleic acids by simple mixing in aqueous buffer. This delivery system serves as an excellent platform for enhanced loading and delivery of genetic material for biomedical research and therapeutic applications with a unique capability to enhance intracellular delivery of nucleic acids to immune cells, which are notoriously difficult to transfect.

Specifically, the nanocarrier platform described herein to deliver polynucleotides is nontoxic, and allows for efficient in vitro transfection of cells, in particular, immune cells. This in vitro transfection of cells is allowed to take place in the presence of serum, and provides superior transfection as compared to the current lipofectamine standard for cell transfection, which is toxic and has been notorious for difficulty in transfecting immune cells. Lipofectamine requires special serum free medium and results in toxic effects and low cell viability after transfection. Immune cells are very sensitive to their medium conditions, thus the ability of the present methods and the presence nanocarriers to be able to deliver polynucleotides in the presence of serum is a significant advantage of the PPDP described herein, in addition to the low toxicity.

As described in the Examples, the optimized PPDP construct transfected macrophages, fibroblasts, dendritic cells, and T cells more efficiently and with less toxicity than the leading Lipo2K reagent, regardless of size of the polynucleotide and under standard culture conditions in the presence of serum. Despite their potential, cationic peptide dendrimers form complexes with nucleic acid primarily through electrostatic interactions, which are generally unstable in serum when used alone and have potential cytotoxicity concern, limiting their application in gene therapy.

The present disclosure provides PEG-b-PPS-linker-DP synthetic polymers, which are capable of producing nanostructures that can be used for polynucleotide delivery. The polymers PEG-b-PPS-linker-DP comprise poly(ethylene glycol)-block-poly(propylene sulfide) copolymer (PEG-b-PPS) conjugated with a dendritic-specific branched cationic peptide (DP). Suitable linkers can include, but are not limited to, for example, 1) covalent bonds 2) ionic bonds or sensitive to 3) enzymatic degradation/proteolysis 4) pH 5) temperature 6) light 7) ultrasound 8) salt concentration 9) surfactants 10) oxidation 11) hydrolysis. Suitable linkers and methods of linking are known in the art. A linker group typically has two ends, wherein one of the ends comprises a substrate (DNA) attaching group and wherein the other of the ends comprises a polymer attaching group, wherein the polymer attaching group. The present invention is not limited to any particular linker group, Indeed, the use of a variety of linker groups is contemplated. including, but not limited to, alkyl, ether, polyether, alkyl amide groups or a combination of these groups. The present invention is not limited to the use of any particular substrate (DNA) attaching group or polymer attaching groups as they are known in the art. It is contemplated that the present invention may use of a variety of polymer attaching groups, including, but not limited to amine, hydroxyl, thiol, carboxylic acid, ester, amide, epoxide, isocayanate, and isothiocyanate groups. In alternative embodiments the linker includes a trityl moiety, an ester moiety. or a CDM (carboxylated dimethyl maleic acid) moieties. As will be appreciated by one of skill in the art, the alternative linker moieties can used in place of the disulfide linker described herein. For convenience of drafting, the specification will be primarily focused on disulfide linkers but it should be appreciated that the alternative moieties can be substituted therewith where applicable.

In a preferred embodiment, the linker is preferably a disulfide bond (ss), (e.g., PEG-b-PPS-ss-DP).

Poly(ethylene glycol)-block-poly(propylene sulfide) copolymers (PEG-b-PPS) can be prepared via known methods, for example those described in Allen, S. et al., Facile assembly and loading of theranostic polymersomes via multi-impingement flash nanoprecipitation J. Control. Release 2017. 262: p. 91-103 and in U.S. Pat. No. 10,633,493, each of which is incorporated herein by reference in its entirety with regard to the method of preparing the copolymers. An exemplary synthesis is described in the Examples. For example, the PEG-b-PPS are prepared via the anionic ring-opening polymerization of propylene sulfide initiated by PEG thioacetate and end-capped with PEG mesylate. The PEG-b-PPS are purified by precipitation in methanol.

To obtain the PEG-b-PPS-linker-DP polymers described herein, the PEG-b-PPS are conjugated to a dendritic-specific branched cationic peptide (DP) via a linker (e.g., disulfide bond) as described in the examples.

“Dendritic peptides,” “DP” or “branched cationic peptides” are peptides with a three-dimensional (3D) architecture with multiple functional groups. Dendritic peptides are branched oligocationic peptides that differ in the number and type (lysine, arginine, ornithine) of cationic amino acid. The dendritic peptide of the present invention has three generations and each unit composed of positively charged arginine (R) for interaction with nucleic acids, histidine (H) with buffering capacity, and lipophilic leucine (L) with membrane-binding ability to facilitate endosomal escape and lysine for functional unit branching. These branched peptides show better encapsulation and transfection efficiency for gene delivery compared with linear peptides. A variety of parameters can affect the activity of dendritic peptides, including generations based on the layer of peptide branching, molecular weight, functional or branching units, and charge distribution. In one embodiment, the dendritic peptide conjugated to the PEG-b-PPS is {[(RHL)2-KRHL]2-KRHL}2-KC—NH2 (SEQ ID NO: 1). In some embodiments, the PEG-b-PPS is conjugated to the DP via disulfide exchange. In some embodiments, the PEG-b-PPS to DP ratio is from 1:1 to 1:1000, preferably about 1:1 to about 1:500. In some embodiments, the PEG-b-PPS to DP ratio is from 1:1, 1:2, 1:10, 1:20, 1:50, 1:75, 1:100, 1:120, 1:200, 1:500, 1:750, etc. including all ratios inbetween.

The term “amino acid” refers to natural amino acids, unnatural amino acids, and amino acid analogs, all in their D and L stereoisomers, unless otherwise indicated, if their structures allow such stereoisomeric forms. Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidine carboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

The nanocarriers described herein can comprise PEGₘ-b-PPSₙ linked to the DP, wherein m and n are both integers each selected from 1-500, alternatively about 2-300, alternatively 10-250. Specific m and n can be selected to provide the specific ration or PEG and PPS to provide the specific nanostructure desired (e.g., polymersome, bicontinous nanospheres, micelles, filomicelles etc. as described herein). In some embodiments, the linker is a disulfide bond (-ss-) but it is contemplated that any linker capable of binding the peptide to the polymer can be used.

The PEG-b-PPS-linker-DP polymers can be characterized for size distribution via dynamic light scattering (DLS) and nanoparticle tracking analysis (NTA), and for morphology via cryogenic transmission electron microscopy (cryoTEM). Therapeutic agent loading and encapsulation efficiencies can be characterized via liquid chromatography mass spectrometry.

A variety of types of PEG-b-PPS-linker-DP polymers can be prepared by varying the degree of propylene sulfide polymerization, oxidation or branching. For example, nanocarriers may be in the form of polymersomes (PEG weight fraction of about 0.25 to about 0.45), micelles (PEG weight fraction above 0.45), biocontinuous nanospheres (PEG weight fraction below 0.25), filomicelles (PEG weight fraction of about 0.35 to about 0.45), polypropylene sulfone nanogels (above 90% oxidized PPS homopolymer), or polymersomes assembled from branched raft polymerized poly(oligo(ethylene glycol) methyl ether methacrylate)-b-poly(oligo(propylenesulfide) methacrylate) (POEGMA-POPSMA¹⁻⁵ In some embodiments, the block copolymer has a PEG weight fraction of about 0.25. One skilled in the art would be able to determine proper weight fractions and may vary from the examples provided herein but still be within the scope of the invention.

In some embodiments, the PEG-b-PPS-linker-DP polymers are polymersomes having an aqueous core and hydrophobic and hydrophilic regions of the lipid bilayer surrounding the aqueous core. The polymersome PEG-b-PPS-linker-DP polymers can have a PEG weight fraction of about 0.25 to about 0.80. The polymersome PEG-b-PPS-linker-DP polymers may have a diameter of about 10 nm to about 300 nm, alternatively from about 30 nm to about 150 nm in diameter, alternatively from about 30 nm to about 60 nm, alternatively from about 60 nm to about 90 nm, alternatively from about 100 nm to about 150 nm in diameter. In some embodiments, the PEG-b-PPS-linker-DP nanostructure is a bicontinuous nanosphere (BCN) characterized by two continuous phases; (i) a cubic lattice of aqueous channels that traverse (ii) an extensive hydrophobic interior volume. Based on small angle X-ray scattering (SAXS) analysis, BCN have primitive type cubic internal organization (Im3m) as confirmed by Bragg peaks with relative spacing ratios at 2, 4, and 6. BCNs are the polymeric equivalent of lipid cubosomes and are lyotropic. BCN can incorporate both hydrophobic and hydrophilic therapeutic agents. BCNs can be prepared via known methods, for examples those described in Allen, S. et al. Benchmarking bicontinuous nanospheres against polymersomes for in vivo biodistribution and dual intracellular delivery of lipophilic and water soluble payloads. ACS Appl. Mater. Interfaces 2018, 10, 40, 33857-33866, which is incorporated herein by reference in its entirety regarding structure and characteristics for the continuous nanosphere structures. In some embodiments, the linker is a disulfide bond (PEG-b-PPS-ss-DP).

In some embodiments, the PEG-b-PPS-linker-DP nanostructure is a micelle or a filomicelle having a hydrophobic/lipophilic core and a hydrophilic exterior. Micelle or filomicelle PEG-b-PPS-ss-DP nanostructures have a spherical morphology and are typically smaller (e.g., less than 50 nm) than polymersomes and the hydrophobic core can be loaded with a nucleic acid. The micelles suitably have a PEG weight fraction of about 0.35 to about 0.45. Micelles or filomicelles can be prepared via known methods, for example those described in Karabin, N. B., Allen, S., Kwon, I-R. et al. Sustained micellar delivery via inducible transitions in nanostructure morphology. Nat Commun 9, 624 (2018), which is incorporated herein by reference.

Other suitable preparation methods of the PEG-b-PPS-linker-DP nanostructure disclosed herein can be prepared via known methods, e.g., Du, F., et al., (2019): Homopolymer Self-Assembly via Poly(propylene Sulfone) Networks. ChemRxiv. Preprint; Du F. et al., Sequential intracellular release of water-soluble cargos from Shell-crosslinked polymersomes. J Control Release. 2018; 282:90-100; and Yi S., et al, Tailoring Nanostructure Morphology for Enhanced Targeting of Dendritic Cells in Atherosclerosis. ACS Nano. 2016; 10(12):11290-11303, each of which are incorporated herein by reference in their entirety.

The PEG-b-PPS polymers provide both hydrophobic moieties of PPS to stabilize the nanostructure and hydrophilic PEG corona to enhance cellular uptake and decrease toxicity. The integration of a bioreducible disulfide bond between PPS and DP improves gene delivery efficiency, due to the improved endosomal escape and cargo release in the reductive intracellular environment.

The disclosure provides nanocarrier system comprising a polynucleotide to be delivered to a cell. The nanocarrier system comprises PEG-b-PPS polymers as described herein conjugated to a DP and forming a structure capable of delivering the polynucleotide to the cell, more preferably delivering the polynucleotide to the nucleus of the cell.

The nanocarrier system described herein can be use for in vitro transfection of cells, particularly immune cells. Suitably, the transfection can be carried out in the presence of serum. Further the nanocarrier systems described herein are non-toxic to the cells. In some embodiments, the nanocarrier system results in a high delivery efficiency of the polynucleotide, specifically when compared to methods of the art such as lipofectamine.

The nanocarrier systems and methods of use described herein are non-toxic to the cells the polynucleotide is being delivered. The term “non-toxic” refers to the ability of the nanocarrier system to no cause apoptosis or cell death when incubated or put into contact with the host cells. Suitably, non-toxic system refers to the ability to retain a majority of the cells being contacted with the nanocarrier system viable and able to incorporate the polynucleotide. Suitably, the term non-toxic refers to not causing a statistically significant decrease in cell viability as assessed by live/dead or metabolic assays (e.g., but not limited to MMT assay) which are known in the art.

The nanocarriers disclosed herein may also be incorporated into pharmaceutical compositions. The disclosed nanocarriers or pharmaceutical compositions comprising the same may be used in methods of gene therapy in a subject in need thereof. The pharmaceutical compositions may further comprise one or more pharmaceutically acceptable excipients. The pharmaceutically acceptable excipients will be dependent on the mode of administration to be used. Suitable modes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the disclosed pharmaceutical compositions are administered parenterally. In some embodiments, parenteral administration is by intrathecal administration, intracerebroventricular administration, or intraparenchymal administration. In particular embodiments, the disclosed pharmaceutical compositions are administered subcutaneously. In particular embodiments, the disclosed pharmaceutical compositions are administered intravenously. The disclosed pharmaceutical compositions herein can be administered as the sole active agent or in combination with other pharmaceutical agents such as other agents used in the treatment of genetic disease in a subject.

The amount of the disclosed nanocarriers or pharmaceutical compositions comprising the same to be administered is dependent on a variety of factors, including the severity of the condition, the age, sex, and weight of the subject, the frequency of administration, the duration of treatment, and the like. The disclosed nanocarriers or pharmaceutical compositions may be administered at any suitable dosage, frequency, and for any suitable duration necessary to achieve the desired therapeutic effect, i.e., to treat genetic disease. The disclosed nanocarriers or pharmaceutical compositions may be administered once per day or multiple times per day. Alternatively, and preferably, the nanocarriers or pharmaceutical compositions may be administered once per week for at least 2 weeks. In other examples, the nanocarriers or pharmaceutical compositions may be administered once per day, twice per day, or three or more times per day. The disclosed nanocarrier or pharmaceutical compositions may be administered daily, every other day, every three days, every four days, every five days, every six days, once per week, once every two weeks, or less than once every two weeks. The nanocarriers or pharmaceutical compositions may be administered for any suitable duration to achieve the desired therapeutic effect, i.e., treat the genetic disease. For example, the nanocarriers or pharmaceutical compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year.

Any suitable dose of the disclosed nanocarriers or pharmaceutical compositions comprising the same may be used. Suitable doses will depend on the therapeutic agent, intended therapeutic effect, body weight of the individual, age of the individual, and the like. In general, suitable dosages of the disclosed nanocarriers or pharmaceutical compositions comprising the same may range from about 0.025 mg nanocarrier/kg body weight to 200 mg nanocarrier/kg body weight. For example, suitable dosages may be about 0.025 mg/kg, or 0.03 mg/kg, or 0.05 mg/kg, or 0.10 mg/kg, or 0.15 mg/kg, or 0.30 mg/kg, to 0.5 mg/kg, or 0.75 mg/kg, or 1.0 mg/kg, or 1.25 mg/kg, or 1.5 mg/kg, or 1.75 mg/kg, or 2.0 mg/kg. In some embodiments, the suitable doses may be 1 mg nanocarrier/kg body weight, or 3 mg/kg, or 5 mg/kg, or 10 mg/kg, or 25 mg/kg, or 50 mg/kg, or 75 mg/kg, or 100 mg/kg, or 125 mg/kg, or 150 mg/kg, or 175 mg/kg, or 200 mg/kg.

In some embodiments, the pharmaceutical composition or nanocarrier may be administered intravenously.

Delivery System

Also described herein is a nanocarrier system for delivering nucleic acids to a cell. The delivery system comprises a nanostructure comprising (a) poly(ethylene glycol)-block-poly(propylene sulfide) copolymer (PEG-b-PPS, as described herein) conjugated with a dendritic-specific branched cationic peptide (DP) (i.e. through a linker), particularly, via a disulfide bond (PEG-b-PPS-ss-DP) and (b) a nucleic acid. The nucleic acid is selected from the group consisting of DNA and RNA. In some embodiments, the nucleic acid is DNA. In some embodiments, the nucleic acid is DNA encoding a gene product or a protein of interest. In some embodiments, the nucleic acid is DNA and the DNA is a plasmid DNA, a DNA construct, or a polynucleotide sequence encoding a protein, peptide or fragment thereof of interest. The inventors have surprisingly found that encapsulation of nucleic acids can be achieved by using a nanostructure of the PEG-b-PPS-linker-DP (PEG-b-PPS-ss-DP) described herein. In preferred embodiments, the nanocarriers are used for in vitro delivery.

The delivery nanocarrier system of the present disclosure has a lower cytotoxicity compared to commercially available transfection agents such as Lipo2K and PEI. Further, the delivery system disclosed herein shows no toxic effect in dendritic cells or macrophages compared an unconjugated DP. Without desire to be bound to any theory, it is believed that the PEG coating on the PEG-b-PPS-linker-DP polymers may suppress the potential toxicity of the DP.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA(+)), minus strand RNA (RNA(−)), synthetic RNA, genomic DNA (gDNA), PCR amplified DNA, complementary DNA (cDNA), synthetic DNA, or recombinant DNA. The polynucleotides preferably encode a protein, peptide or therapeutic target of interest. In some embodiments, the polynucleotide may be a vector or construct. In some embodiments, the polynucleotide may be a DNA vector. As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). Suitable vectors for use with the present invention comprise a promoter operably connected to a polynucleotide sequence encoding a protein or peptide of interest. The term vector encompasses “plasmids”, the most commonly used form of vector. Plasmids are circular double-stranded DNA loops into which additional DNA segments (e.g., those encoding peptides) may be ligated. In some embodiments, the vector is a mini-circle DNA (mcDNA) vector. Mini-circle DNA vectors are episomal DNA vectors that are produced as circular expression cassettes devoid of any bacterial plasmid DNA backbone. See, e.g. System Biosciences, Mountain View CA, MN501A-1. Their smaller molecular size enables more efficient transfections and offers sustained expression over a period of weeks as compared to standard plasmid vectors that only work for a few days. In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10,000, or at least 15,000 or more nucleotides in length, either ribonucleotides or deoxynucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc. 151, 152, 153, etc. 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence.

In some embodiments, the polynucleotide may be a gene or a cDNA encoding a protein or a polynucleotide encoding a DNA or an RNA sequence that encodes a therapeutic agent. In some embodiments, the polynucleotide may be a gene encoding a protein of interest for therapy.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide. In particular embodiments, the term “gene” refers to a cDNA. cDNA are polynucleotides the encode for a protein and do not contain introns and can be artificially produced.

The delivery system can be prepared/loaded, for example, by mixing under physiological conditions the nucleic acid and nanocarrier (i.e. PEG-b-PPS-linker-DP).

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

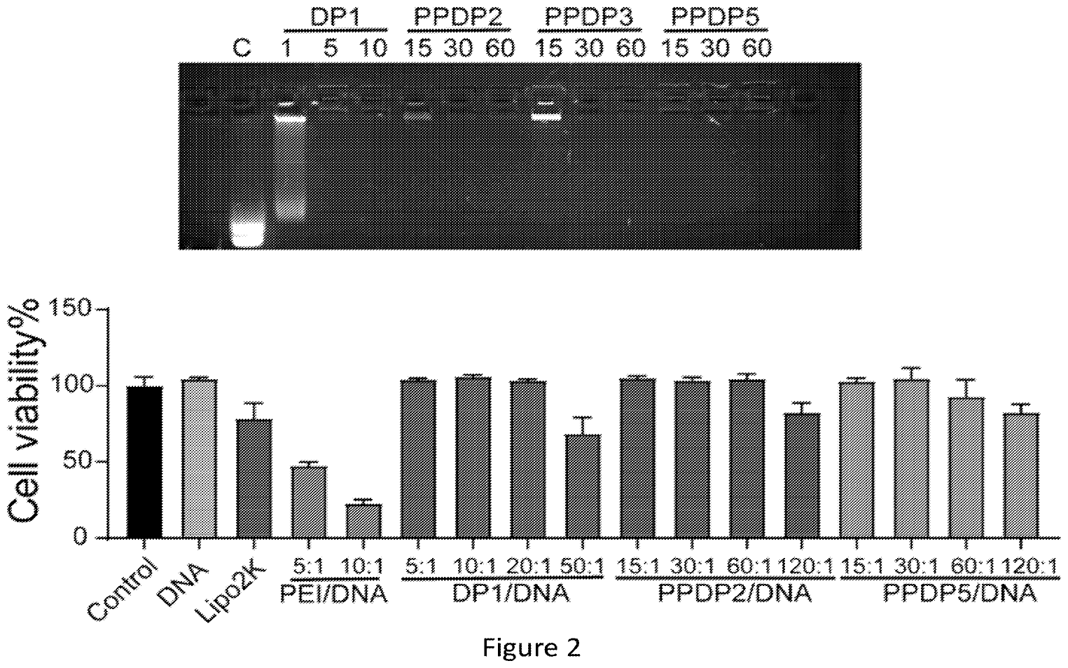

The delivery system may comprise any suitable mass ratio of PEG-b-PPS-linker-DP:nucleic acid necessary to achieve the desired effect. For example, the delivery system may comprise a mass ratio (w/w) of PEG-b-PPS-ss-DP:nucleic acid of 5:1 to 130:1. For example, the mass ratio may be 5:1, 10:1, 15:1, 30:1, 50:1, 75:1, 100:1, 115:1, 120:1, or 130:1. In particular embodiments, the mass ratio of PEG-b-PPS-ss-DP:nucleic acid is from 15:1 to 120:1. The disclosed delivery system containing PEG-b-PPS offers advantages over current delivery methods, including (i) no cytotoxicity, (ii) ability to load large cargoes such as plasmids, (iii) ability to load small cargoes such as nucleotide adjuvant cyclic guanosine monophosphate-adenosine monophosphate (cyclic GMP-AMP or cGAMP), (iv) controllable surface chemistry of the nanocarrier to specify and avoid cellular and biochemical interactions, and (v) both passive and active means of triggering payload release. Cytotoxicity is a primary concern in the development of gene delivery vectors for biomedical applications. The delivery system disclosed here showed no toxic effect with the PPDP/DNA under 60:1, while minimal toxic effect when the PPDP/DNA up to 120:1 (e.g., cell viability >80% at PPDP/DNA=120/1) (FIG. 2). In contrast, the unconjugated DP results in lower cell viability even at a lower peptide/DNA weight ratio of 50:1 (DP/DNA=50/1). The cytotoxicity of the nanostructures formed with PEG-b-PPS-linker-DP polymers was much less than the currently available commercial transfection agents Lipo2K (78% viability) and PEI (47% viability for PEI/DNA=5:1, and 22% viability for PEI/DNA=10:1). The Inventors have surprisingly found that the PEG coating found in the nanostructures of PEG-b-PPS-linker-DP could suppress the potential toxicity of DP.

The polymers used in the PEG-b-PPS-linker-DP, poly(ethylene glycol) and poly(propylene sulfide) have been widely proven to be inert. Suitably, the polymer is PEGₘ-b-PPSₙ-linker-DP wherein m and n are integers each selected from 1-500. In some embodiments, the linker is disulfide bond (-ss-).

In a further embodiment, the present disclosure provides a vaccine composition comprising a carrier, a DNA antigen or immunogen, and an adjuvant. The carrier may a nanostructure of the PEG-b-PPS-linker-DP polymers disclosed herein. Without being bound by any theory, the use of the PEG-b-PPS-linker-DP nanostructures as carriers in a vaccine composition may enhance the delivery of the DNA antigen or immunogen to the cell of interest.

Suitable adjuvants are known in the art and include, but are not limited to, threonyl muramyl dipeptide (MDP) (Byars et al., 1987), Ribi adjuvant system components (Corixa Corp., Seattle, Wash.) such as the cell wall skeleton (CWS) component, Freund's complete adjuvants, Freund's incomplete adjuvants, bacterial lipopolysaccharide (LPS; e.g., from E. coli), or a combination thereof. A variety of other well-known adjuvants may also be used with the methods and vaccines of the invention, such as aluminum hydroxide, saponin, amorphous aluminum hydroxyphosphate sulfate (AAHS), aluminum hydroxide, aluminum phosphate, potassium aluminum sulfate (Alum), and combinations thereof. Cytokines (.gamma.-IFN, GM-CSF, CSF, etc.), lymphokines, and interleukins (IL-1, IL-2, IL-3, IL-4, IL-5, IL-6, IL-7, IL-8. IL-9, IL-10, IL-11, IL-12, IL-13, IL-14, IL-15, IL-16, IL-17, 11-18, 11-19, IL-20, IL-21, and 11-22) have also been used as adjuvants and/or supplements within vaccine compositions and are contemplated to be within the scope of the present invention. For example, one or more different cytokines and/or lymphokines can be included in a composition comprising one or more peptides or a vaccine of the invention.

The term “antigen” or “immunogen” as used herein refers to a compound or composition comprising a peptide, polypeptide or protein which is “antigenic” or “immunogenic” when administered (or expressed in vivo by an administered nucleic acid, e.g., a DNA vaccine) in an appropriate amount (an “immunogenically effective amount”), i.e., capable of inducing, eliciting, augmenting or boosting a cellular and/or humoral immune response either alone or in combination or linked or fused to another substance (which can be administered at once or over several intervals). An immunogenic composition can comprise an antigenic peptide of at least about 5 amino acids, a peptide of 10 amino acids in length, a polypeptide fragment of 15 amino acids in length, 20 amino acids in length or longer. The immunogen can be recombinantly expressed from a vaccine vector, which can be naked DNA comprising the immunogen's coding sequence operably linked to a promoter, e.g., an expression cassette.

### Methods

The present disclosure also provides in some embodiments methods of delivering a nucleic acid to a cell, the method comprising contacting or administering to the cell the delivery system disclosed herein. In particular embodiments, the nucleic acid is DNA. In particular embodiments, the cell is an immune cell. In particular embodiments, the cell is a dendritic cell. In another embodiment, the cell is a macrophage.

In a further embodiment, a non-toxic in vitro method of delivering a polynucleotide to a cell is provided. The method comprises contacting the cell in culture with a nanocarrier system described herein (e.g., comprising a nanocarrier comprising PEGₘ-b-PPSₙ-covalently linked to a dendritic peptide (DP) and a polynucleotide); and culturing the cells for a sufficient time to allow the cell to uptake the nanocarrier system and deliver the polynucleotide to the cell. In some embodiment, both the contacting and culturing step are carried out in culture medium comprising serum. This is important for immune cells, including dendritic cells, which are very sensitive to culture conditions.

In some embodiments, the cell is an immune cell. Suitable immune cells are known in the art and include, for example, dendritic cell, macrophage, T cell, B cell, or the like.

A “dendritic cell” or “DC” is the antigen presenting cells of the mammalian immune system. DCs function to process antigen material and present it on their surface to T cells of the immune systems and act as a messenger between the innate and the adaptive immune system. DCs express high levels of the molecules that are required for antigen presentation such as the MHC II, CD80, and CD86 on activation and are highly effective in initiating an immune response. DCs are distributed throughout the body, including the mucosal tissues, where they are found below the epithelial cell barrier. DCs have been found to play roles in progressive decline in adaptive immune responses, loss of tolerance and development of chronic inflammation. Dendritic cells may be present in the normal arterial wall and within atherosclerotic lesions.

The present disclosure also provides in some embodiments methods of transfecting an immune cell to deliver a polynucleotide sequence to the nucleus of the immune cell, the method comprising contacting the immune cell with the system disclosed herein for a sufficient time to deliver the polynucleotide sequence to the nucleus of the immune cell. In some embodiments, the immune cell is in vitro. In some embodiments, the immune cell is in vivo.

The term “transduced” or “transfected” refers to the ability of a exogenous polynucleotide to be introduced to a cell, particularly introduced to the nucleus of a cell. In some embodiments, the polynucleotide is capable of expression of a protein when it is transcribed and translated within the nucleus of the cell.

“Transduced” or “Transfected” cells can, when transduced with a nucleic acid (plasmid) that encodes a protein or comprises a sequence that is transcribed into a transcript of interest, can produce protein and/or transcript. Additionally, such cells when transduced with polynucleotide sequences, such as plasmids that encode a gene of interest that encodes a protein or is transcribed into a transcript of interest, can produce vectors that include the gene that encodes a protein or comprises a sequence that is transcribed into a transcript of interest, which in turn produces vectors of interest. In particular embodiments, the polynucleotide encodes a therapeutic agent.

The present disclosure also provides in some embodiments methods of treating a subject in need of gene therapy, the method comprising administering to the subject an effective amount of the delivery system described herein, where the delivery system comprises a nucleic acid comprising a gene of interest for gene therapy.

In some embodiments, for example, the delivery system comprising the polynucleotide, is administered to (or introduced into) one or more cell or tissue types of interest in order to disrupt or enable regulation of one or more genes of interest, such as a gene of interest or a gene associated with a disease of interest.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human. In some embodiments, the subject is a mammal in need of gene therapy. The term “subject” does not denote a particular age or sex. In one specific embodiment, a subject is a mammal, preferably a human. In a suitable embodiment, the subject is a human in need of gene therapy.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of” and “consisting of” those certain elements.

As used herein, “about” means within 5-10% of a stated concentration range or within 5-10% of a stated number.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit's interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1

Recent progress in genetic engineering offer an unparalleled opportunity for cancer immunotherapy^{[1]}, antiviral therapies^{[2]}, and DNA vaccines^{[3]}, as well as non-medical uses^{[4]}. Notwithstanding a large number of ongoing trials in the area of genome editing, successful gene therapy products are very limited^{[5-7]}. This is largely due to challenges surrounding the effective expression of nucleic acids after delivery into target cells. Additionally, an efficient DNA delivery system must also overcome several obstacles such as protection from enzyme degradation, cellular internalization, escape from endo-lysosomes, and cytosolic cargo release. Plasmid-based gene therapy is a promising transfection strategy that is capable of delivering CRISPR/Cas9^{[8-10]} for stable genome editing using an all-in-one plasmid approach^{[11]}. However, naked plasmid DNA (pDNA) has many obstacles^{[12,13]} for cellular uptake due to its negative charge, susceptibility to enzymatic degradation, large molecular size that limits delivery via viral vectors, and low encapsulation efficiency in synthetic nanotechnologies.

As natural gene delivery vehicles, viruses exhibit unprecedented performance for gene expression; however, various issues^{[14]}, such as insertional mutagenesis^{[15]}, inherent immunogenicity^{[16]}, and pre-existing host antibodies against viral components^{[17]}, can reduce their efficacy and utility in clinical settings^{[18]}. Importantly, the limited DNA-carrying capacity (<8 kb)^{[19]} and manufacturing challenges^{[20-23]} pose additional restrictions for viral-based vectors. Non-viral vectors and synthetic carriers, including lipids, polymers, peptides, and inorganic nanomaterials have therefore attracted increasing attention due to their limited immunogenicity, flexible packaging capacity, and amenability to scalable fabrication methods, and access to diverse mechanisms of intracellular delivery for improved transfection efficiency^{[13,24-26]}. However, existing materials still face challenges such as inefficient endosomal escape, substantial toxicity, and low gene transfection/cellular expression. The issue of low transfection is particularly problematic for certain cell types, particularly immune cells that are highly desired as targets for diverse therapeutic applications including vaccination and cancer immunotherapy. For example, cationic lipids are the most widely used non-viral vectors for nucleic acid delivery in most immortalized cell lines, yet many blood and immune cells remain recalcitrant^{[27]}. Lipid-based technologies aside, many polymeric transfection reagents, such as polyethylenimine (PEI), have long suffered from cytotoxicity concerns^{[28-30]} Thus, there is high demand for alternative technologies that are capable of delivering nucleic acids to difficult-to-transfect cells, especially primary cells and immune cells under their standard culture conditions, without associated increases in toxicity.

Dendritic or branched cationic peptides have a three-dimensional (3D) architecture with multiple functional groups, which makes them well suited for delivering negatively charged nucleic acids^{[31,32]}. It has been reported that dendritic structures can significantly enhance the interaction of peptides with DNA, considerably improve cargo packing, and increase the transfection efficiency in diverse cell types, compared to linear DNA-binding peptides^{[33]}. Notably, a variety of parameters influence the activity and biocompatibility of dendritic peptides, including molecular weight, functionality of branching units, and charge distribution^{[32,34-37]}. In particular, third generation peptide dendrimers with lower molecular weight to charge ratios and charged distributed over the whole dendritic structure have been demonstrated to be the best transfection reagent for DNA delivery^{[36]}. Despite their potential, cationic peptide dendrimers form complexes with nucleic acid primarily through electrostatic interactions, which are generally unstable in serum when used alone and have potential cytotoxicity concerns^{[38]}, limiting their application in gene therapy.

In this example we demonstrate the development a novel gene delivery system employing self-assembling block copolymer nanocarriers engineered for efficient cytosolic delivery of plasmid DNA via incorporation of a cationic dendritic peptide (DP) block (FIG. 1). We have shown poly (ethylene glycol)-block-poly (propylene sulfide) (PEG-b-PPS) nanocarriers have a superior capacity to target macrophages and dendritic cells^{[39-41]}, are capable of delivering diverse payloads intracellularly^{[42-49]}, are non-immunogenic in human blood^{[50]}, and are both non-inflammatory and non-toxic in non-human primates^{[51]}, humanized mice^{[52]}, and diverse mouse models of disease^{[39]}.

This work is motivated by natural viruses possessing capsid proteins that utilize positively charged regions to carry genes, which encapsulate this genetic material within a higher order protein framework/structure that is largely stabilized by non-covalent interactions between hydrophobic regions^{[53-55]}. We demonstrate that the high stability of hydrophobic PPS membranes and volumetric differences between the PEG and dendritic peptide blocks allow sequestering of large genetic elements within the interior of self-assembled PEG-b-PPS nanocarriers.

The dendritic peptide (DP) has three generations and each unit composed of positively charged arginine (R) for interaction with genes, histidine (H) with buffering capacity, and lipophilic leucine (L) with membrane-binding ability to facilitate endosomal escape and lysine for functional unit branching. The PEG-b-PPS polymers can provide not only hydrophobic moieties of PPS to stabilize the nanostructure but also the hydrophilic PEG corona to enhance cellular uptake and decrease toxicity. The integration of a bioreducible disulfide bond between PPS and DP could improve the gene delivery efficiency, due to the improved endosomal escape and cargo release in the reductive intracellular environment.

PEG-b-PPS copolymer was modified with a functional, cationic DP using a cysteine linker (PEGₘ-b-PPSₙ-ss-DP, PPDP) (FIG. 1). Each unit of the DP is composed of lysine for functional unit branching, lipophilic leucine to help bind membranes and facilitate escape from endolysosomal compartments^{[56]}, a histidine residue for its buffering capacity and to assist the disruption of endosomal membranes^{[56]}, and arginine to stably interact with negatively charged DNA. The arginine residues interact with anionic nucleic acids under diverse conditions, since they are positively charged in the extracellular environment (prior to internalization) and at all pH conditions encountered within the endolysosomal pathway following cellular internalization. In addition to the peptide aspects of our technology, the PEG-b-PPS polymers contributes multiple useful features for gene delivery. PEG-b-PPS provides oxidation-sensitivity via its hydrophobic PPS blocks that stabilize the nanostructure and enable disassembly within acidic endolysosomal compartments^{[42,43,46,47,49]}. The hydrophilic methoxy-terminated PEG corona serves to improve biocompatibility and reduce toxicity^{[50,51]} Furthermore, we incorporated a biologically-reducible disulfide bond between the terminus of PPS and the DP to improve release of DP-bound pDNA from PEG-b-PPS nanocarriers within reductive intracellular compartments.

We synthesized a library of self-assembling PPDP polymers and thoroughly examine their functionality as non-viral nanovectors for gene delivery. Cellular transfection experiments were benchmarked against the leading commercial product, Lipofectamine 2000 (Lipo2K). Furthermore, to understand whether our gene delivery platform is compatible with a range of DNA cargo sizes, we examined the transfection efficiency of a small DNA plasmid (S-pDNA; pCMV-DsRed, 4.6 kb) and a large DNA plasmid (L-pDNA; pL-CRISPR.EFS.tRFP, 11.7 kb). After conducting a comprehensive screen of PPDP-mediated transfection performance in macrophages as a model immune cell type, we optimize the formulation of the top performing nanovectors and elucidate the mechanistic details of their functionality in a series of multidisciplinary studies. Finally, the efficacy of the optimal PPDP nanovectors is assessed in NIH 3T3 fibroblasts, as well as a variety of difficult-to-transfect immune cell types, including T cells and primary bone marrow-derived dendritic cells (BMDCs).

PEG-PPS polymers conjugated with DP (PPDP) were assembled into stable nanostructures with plasmid DNA by simple mixing in aqueous buffer. We hypothesized that PPDP might enhance DNA encapsulation via electrostatic interactions in addition to increased hydrophobic effects. To investigate the hydrophobic effects of PPDP on gene transfection, we synthesized a family of PEG-PPS polymers with various hydrophobic ratios and conjugated with DP. Screening studies revealed that the optimized PPDP nanostructure achieved superior efficiency for DNA delivery in a variety of cell types, including primary bone marrow-derived dendritic cells (BMDCs) compared to that of leading commercial products, such as Lipofectamine 2000. Furthermore, our gene delivery system enabled efficient cytosolic delivery of plasmid DNA ranging from 6.4 kb to 11.7 kb. This gene delivery system may, therefore, serve as an excellent platform to enable efficient genetic engineering for biomedical research and therapeutic applications.

### Results and Discussion

PDP Preparation and Characterization

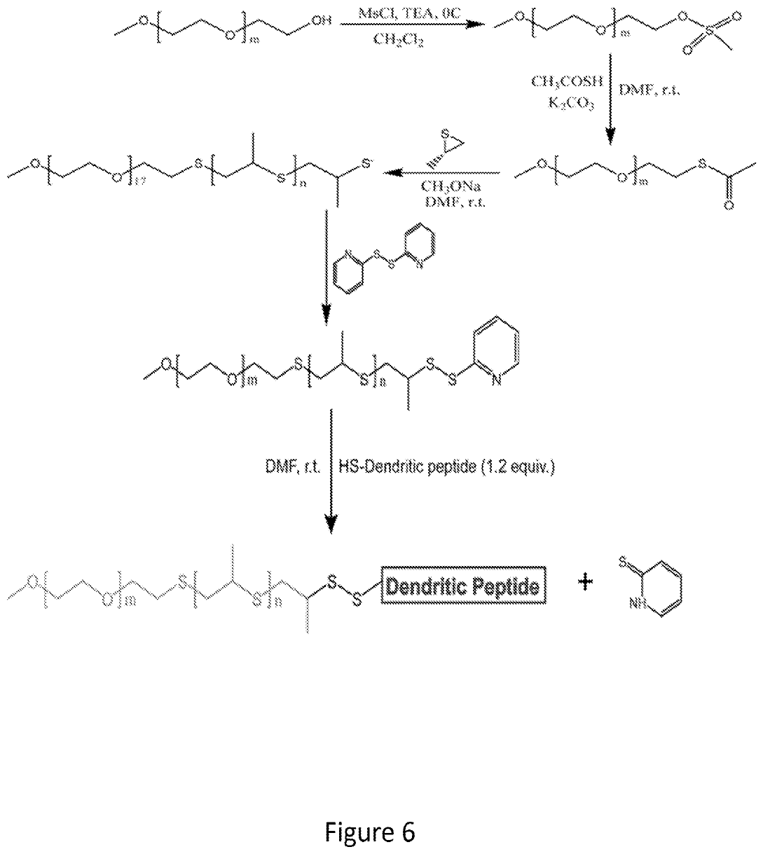

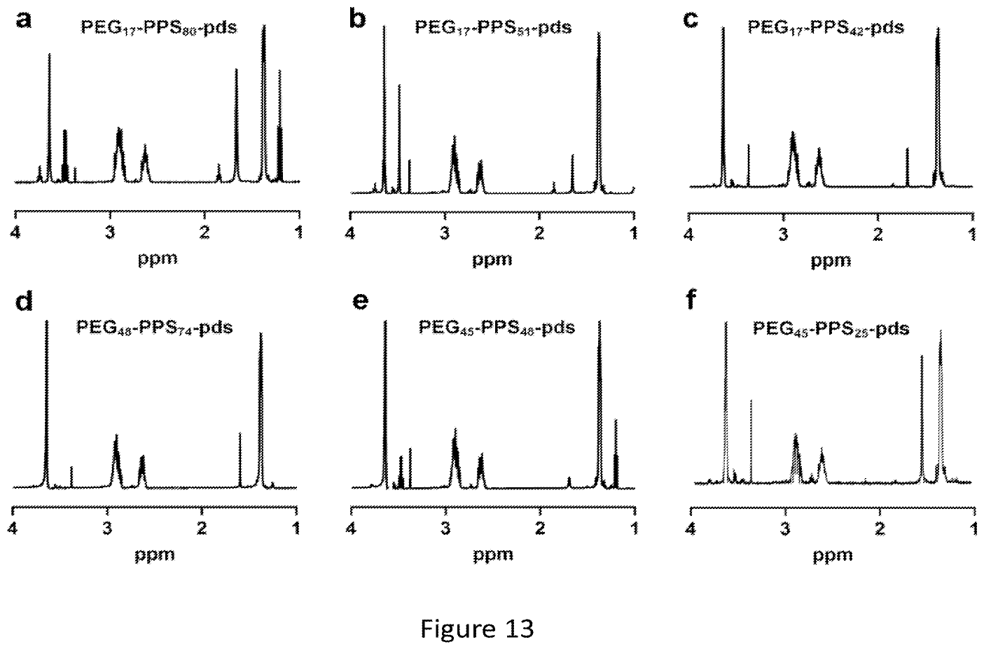

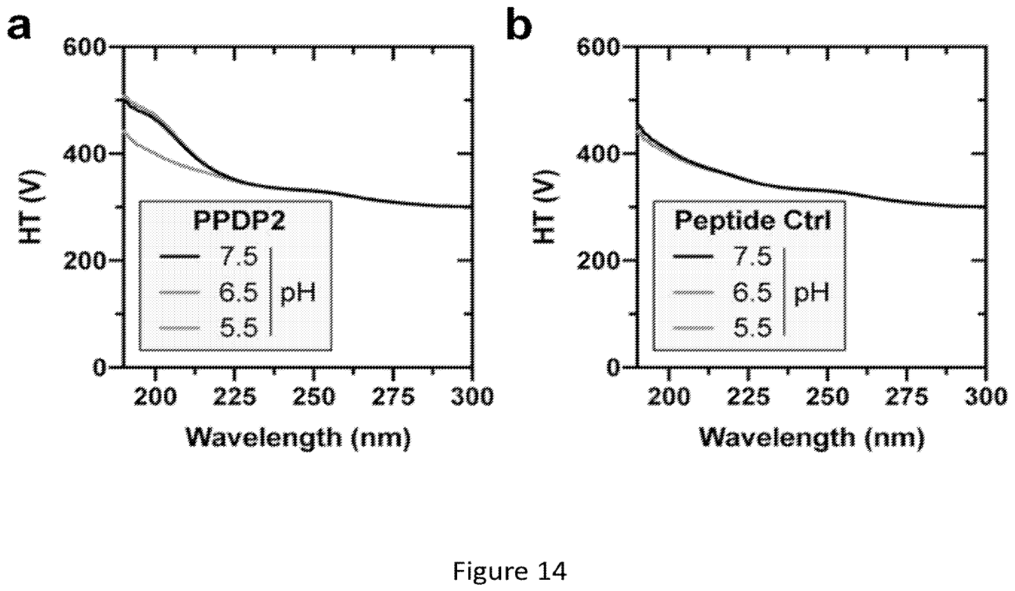

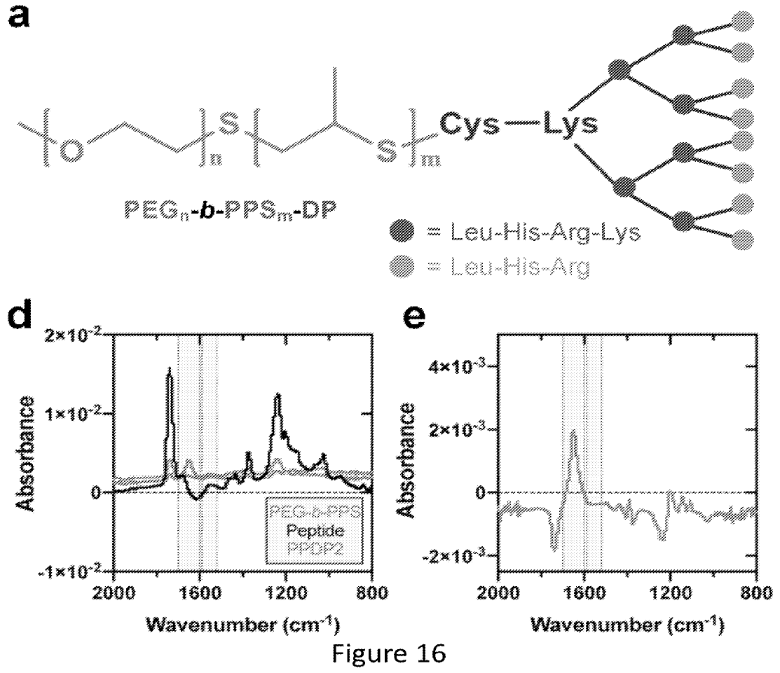

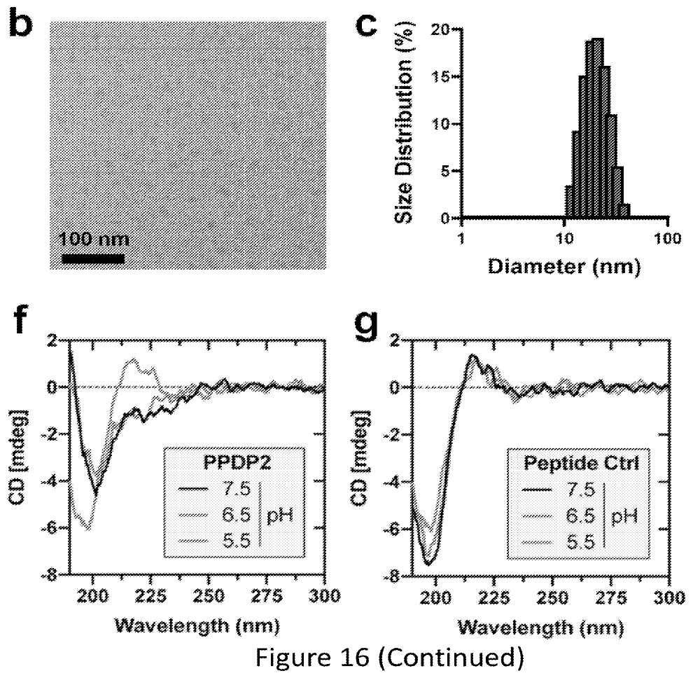

To investigate how the molecular weight and hydrophobicity influence gene delivery in diverse cell types, six PEG-b-PPS di-block copolymers with different PEG molecular weight of 750 or 2000, and various PPS lengths from 20 to 80 via ionic polymerization under strictly anhydrous conditions (FIG. 6). The PEGₘ-b-PPSₙ polymers end-capped with pyridyl disulfide (PEGₘ-b-PPSₙ-pds) were characterized and verified by NMR and GPC (FIG. 13). The cysteine-containing DP was then conjugated with PEGₘ-b-PPSₙ-pds via disulfide exchange reaction (FIG. 16). To simplify nomenclature in this study, PEG-PPS-ss-DP polymers with diverse molecular weight and hydrophobicity were designated from PPDP2 to PPDP7, as shown in Table 1.

TABLE 1
Physicochemical characteristics of PEGₙ-b-PPSₘ-ss-DP (PPDP).
Data are presented as mean ± SD (n = 3).

| | Average | Zeta | |
|---|---|---|---|
| | diameter | potential | Polydispersity |
| Samples | ±SD (nm) | ±SD (mV) | index (PDI) |
| PPDP2 | 19.63 ± 0.27 | 48.03 ± 2.74 | 0.096 |
| (PEG₁₇-PPS₈₀-ss-DP) | | | |
| PPDP3 | 24.02 ± 0.26 | 10.9 ± 0.28 | 0.348 |
| (PEG₁₇-PPS₅₁-ss-DP) | | | |
| PPDP4 | 24.59 ± 1.04 | 14.32 ± 7.34 | 0.292 |
| (PEG₁₇-PPS₄₂-ss-DP) | | | |
| PPDP5 | 24.44 ± 0.46 | 29.47 ± 3.94 | 0.195 |
| (PEG₄₅-PPS₇₄-ss-DP) | | | |
| PPDP6 | 32.11 ± 0.67 | 9.77 ± 0.59 | 0.279 |
| (PEG₄₅-PPS₄₈-ss-DP) | | | |
| PPDP7 | 18.08 ± 0.19 | 16.68 ± 4.99 | 0.201 |
| (PEG₄₅-PPS₂₅-ss-DP) | | | |

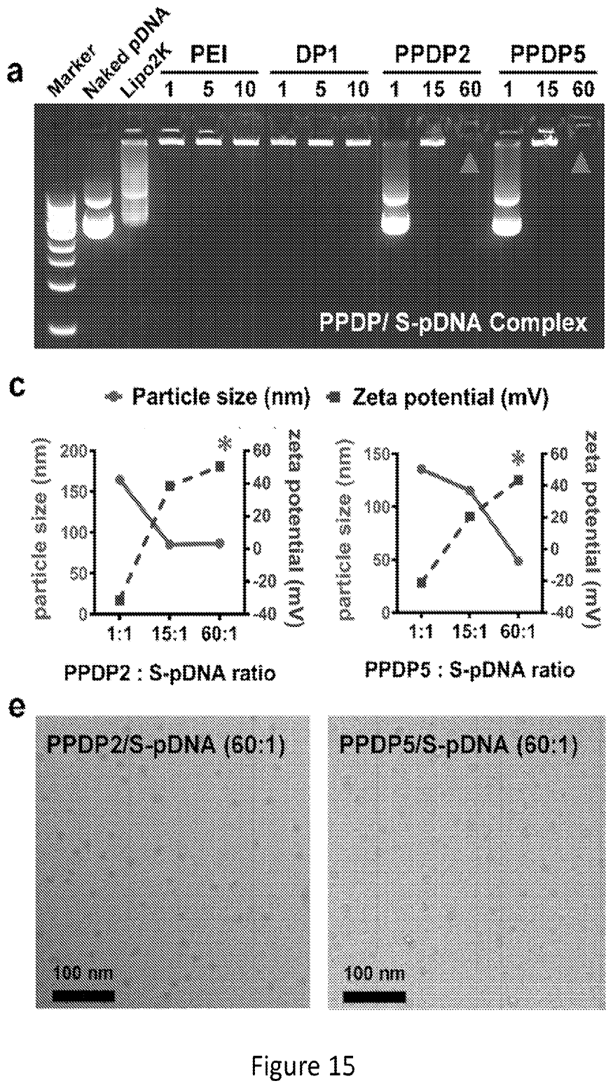

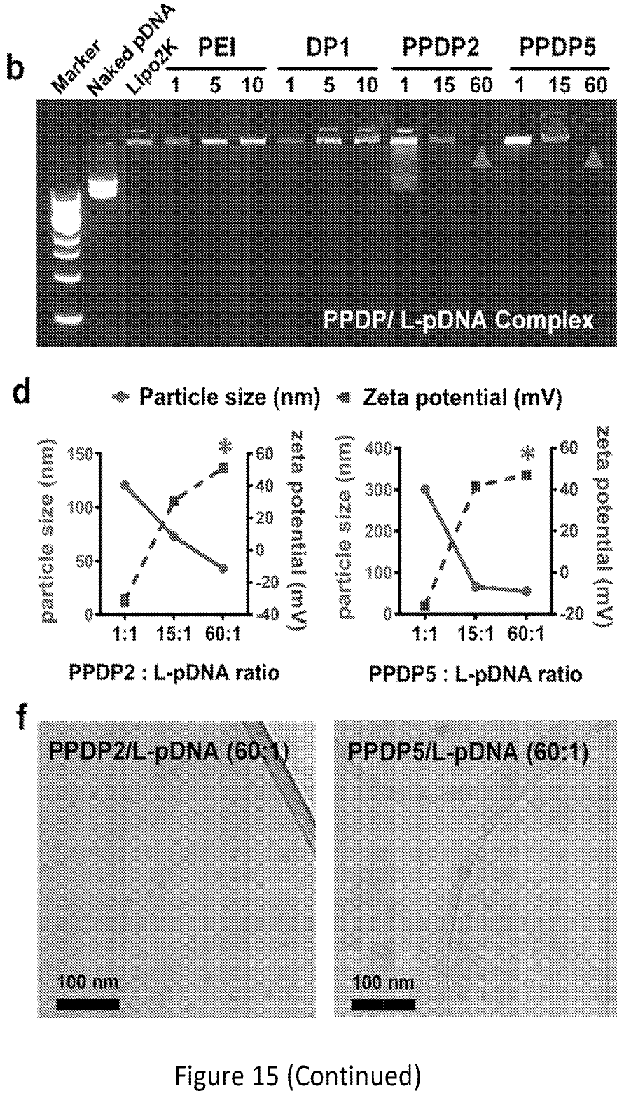

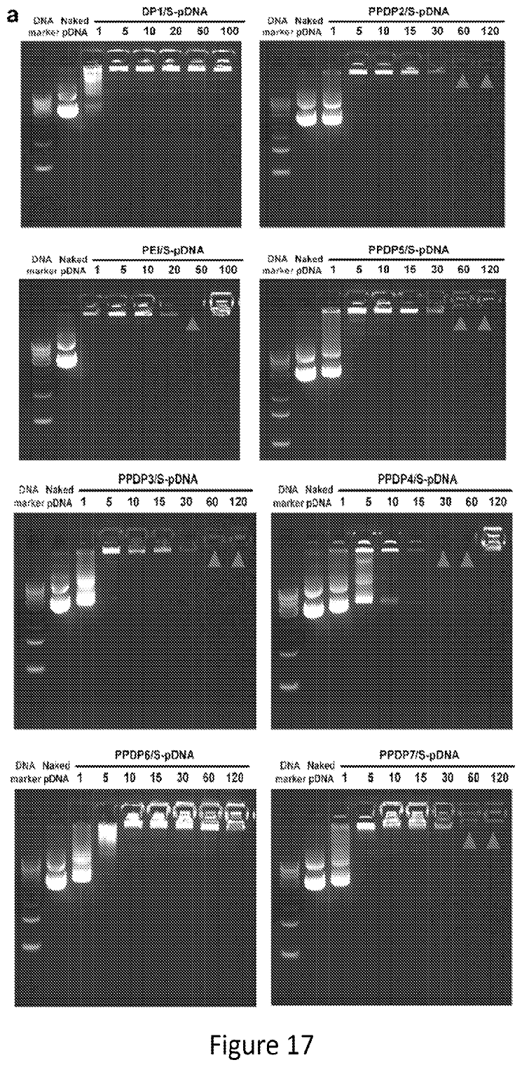

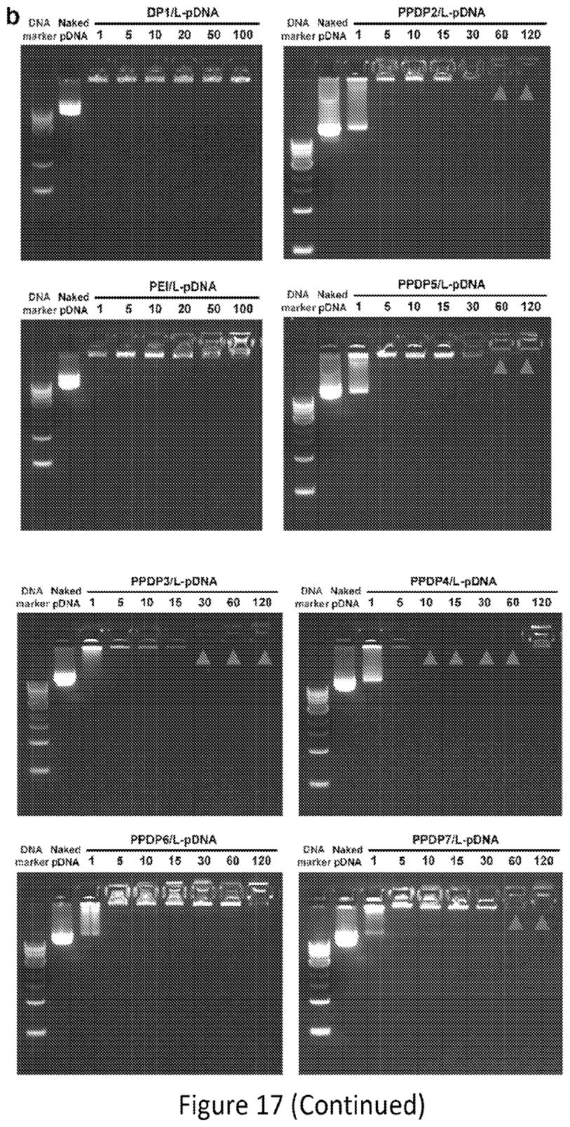

To investigate the DNA encapsulation capabilities of the polymers, DNA-PPDP complexes were firstly formulated by mixing the PPDP solution into the DNA solution at various weight ratios (PPDP:DNA=1:1-120:1) (FIG. 17). The DNA-PPDP complexes formation and stability were evaluated by the gel retardation assay (FIG. 15). In this assay, the stably formed DNA-PPDP complexes would remain in the loading wells, while unbound DNA would migrate down the agarose gel. DP without polymer conjugation (DP1) and a series of PPDP polymers (PPDP2 to PPDP7) were mixed with DNA in PBS at various polymer to DNA weight ratios. The protection of nucleic acids from enzymes/nucleases extracellular or intracellular environment is critical for successful gene delivery. The well-encapsulated DNA can be protected from staining by EtBr. Therefore, the gel retardation assay can also provide information about DNA protection by the nanostructures from the environment. FIG. 2 showed that DNA migration was completely retarded by PPDP nanostructures with the polymer/DNA weight ratio over 15:1. More importantly, the DNA fluorescence progressively decreased with the increase of polymer/DNA ratio, suggesting the enhanced DNA encapsulation with more polymers. Of note, with the same polymer/DNA ratio, DNA-PPDP2 showed the lowest DNA fluorescent intensity in the wells, indicating it provided the best binding capacity and protection of DNA. Other than encapsulation and protection of gene, the size of the DNA-nanovector complex is also essential for cellular uptake (FIGS. 15c and 15d).

The hydrodynamic sizes of DNA-PPDP nanocomplexes were determined by DLS. The PPDP library was self-assembled into stable nanostructures by simple mixing in aqueous solution. The size distribution and zeta potential of PPDP polymers were measured by dynamic light scattering (DLS) analysis. The nanostructures formed from PPDP2-PPDP7 polymers differed in size (from about 20 to 30 nm) and zeta potential (from about 10 to 40 mV) depending on the distinct combination of PEG molecular weight and PPS length of the polymer variant. Furthermore, small angle x-ray scattering (SAXS) performed using synchrotron radiation demonstrated the presence of a spherical core shell morphology.

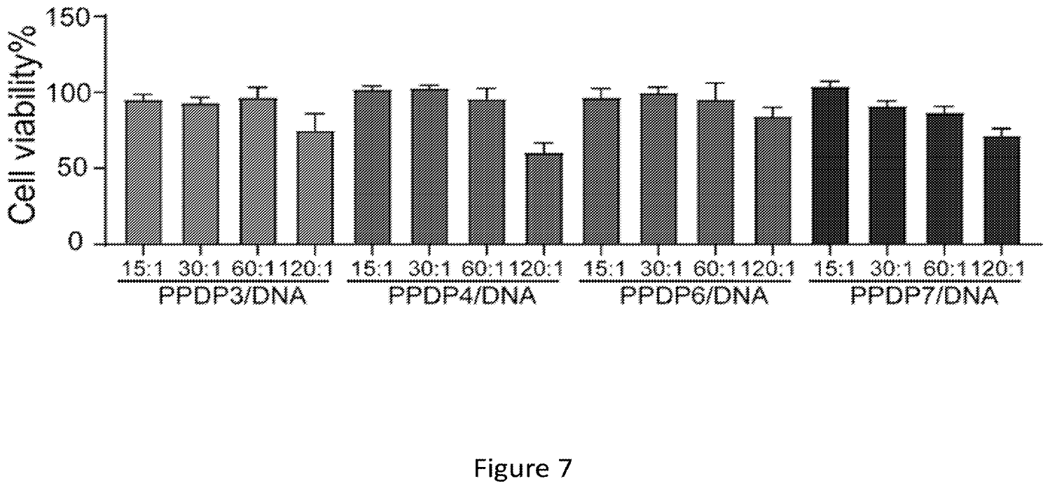

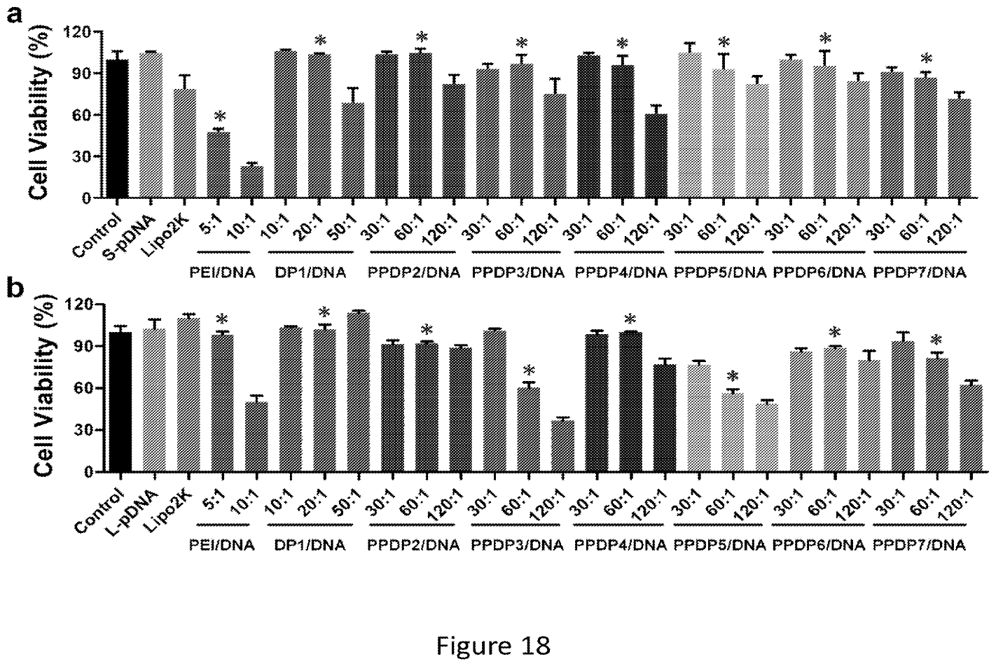

Cytotoxicity is a primary concern in the development of gene delivery vectors for biomedical applications. We have evaluated the cytotoxicity of PPDP nanostructures complexed with DNA in a range of weight ratios in Raw264.7 macrophages. MTT assays demonstrated that DNA-PPDP nanocomplexes showed no toxic effect with the PPDP/DNA under 60:1, while minimal toxic effect when the PPDP/DNA up to 120:1 (e.g., cell viability >80% at PPDP/DNA=120/1) (FIG. 2, FIG. 7, FIG. 18). However, the DP without the conjugation of PEG-b-PPS polymers indicated lower cell viability even at a lower peptide/DNA weight ratio of 50:1. The cytotoxicity of PPDP nanostructures was much less than the commercial transfection agent Lipo2K (78% viability) and PEI (47% viability for PEI/DNA=5:1, and 22% viability for PEI/DNA=10:1). All the data suggested that incorporating PEG coating on PPDP could suppress the potential toxicity of DP.

Gene Delivery with PPDP Nanostructures—Benchmarking PPDP Transfection Performance and Cytotoxicity in Macrophages

PPDP2 and PPDP5 Achieve Significantly Greater Transfection Efficiency and Reporter Protein Expression Levels than Lipo2K

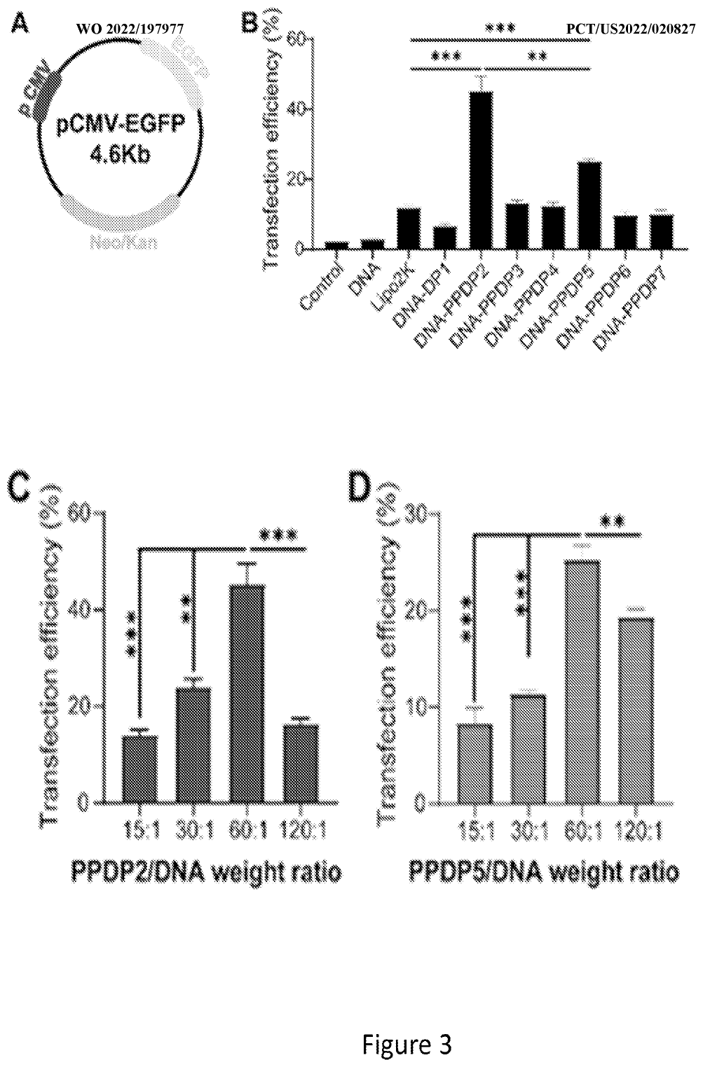

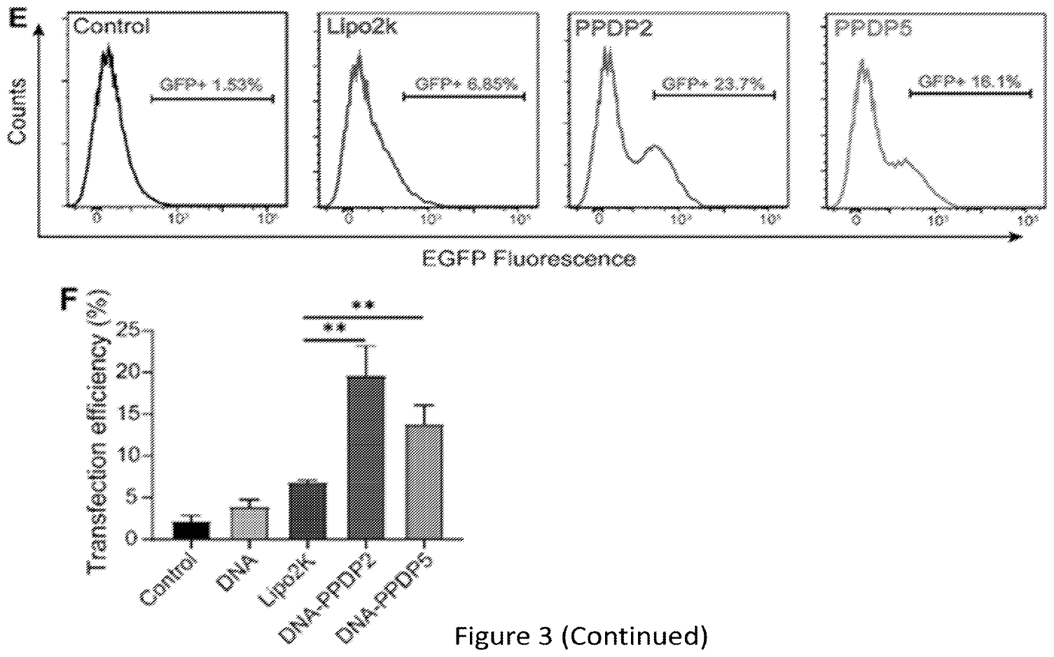

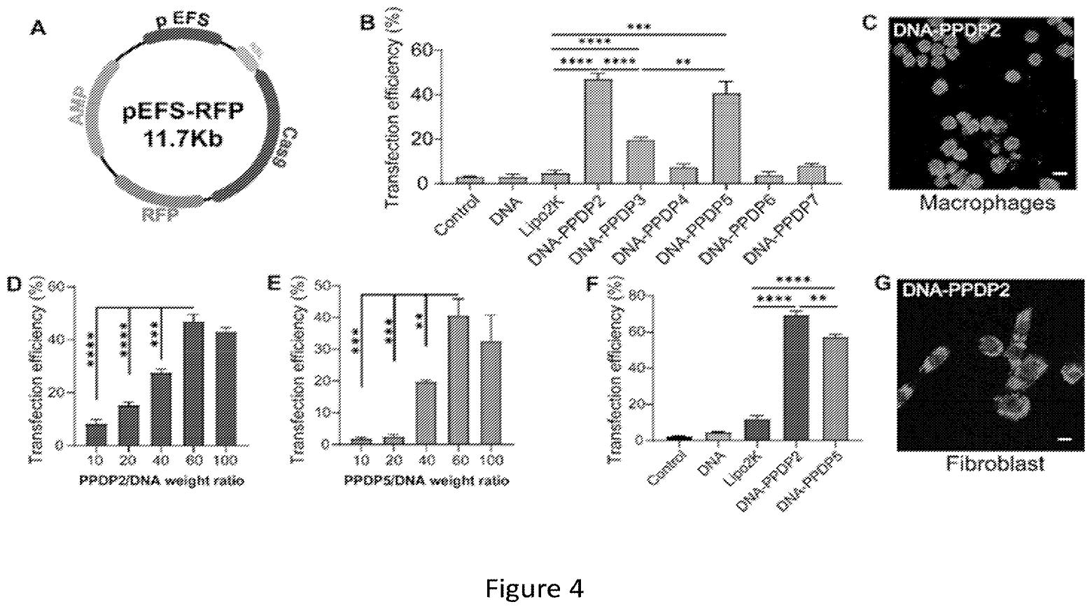

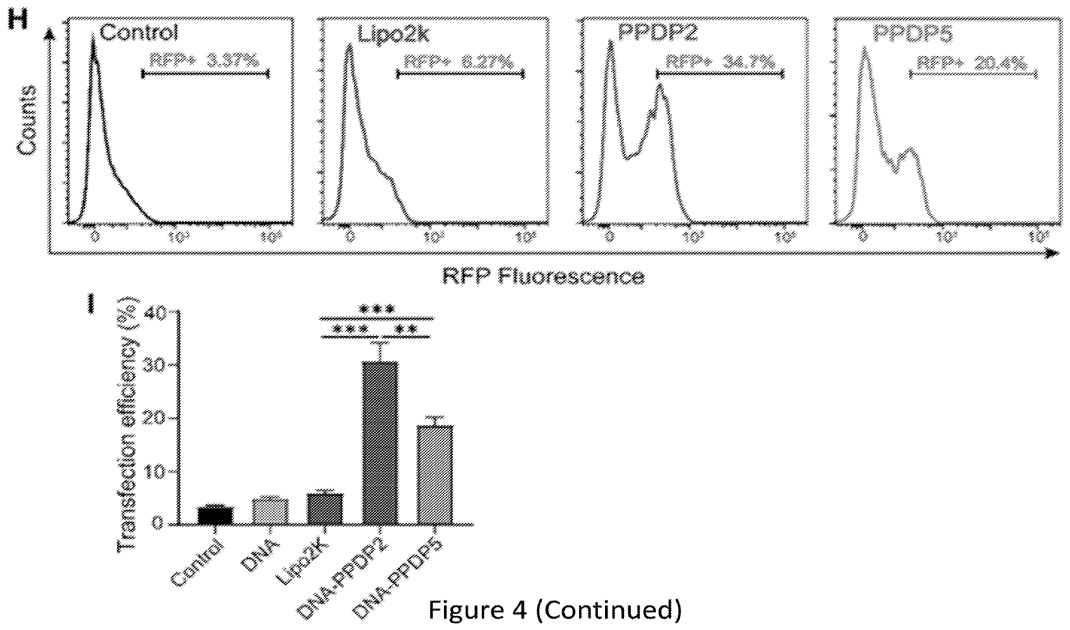

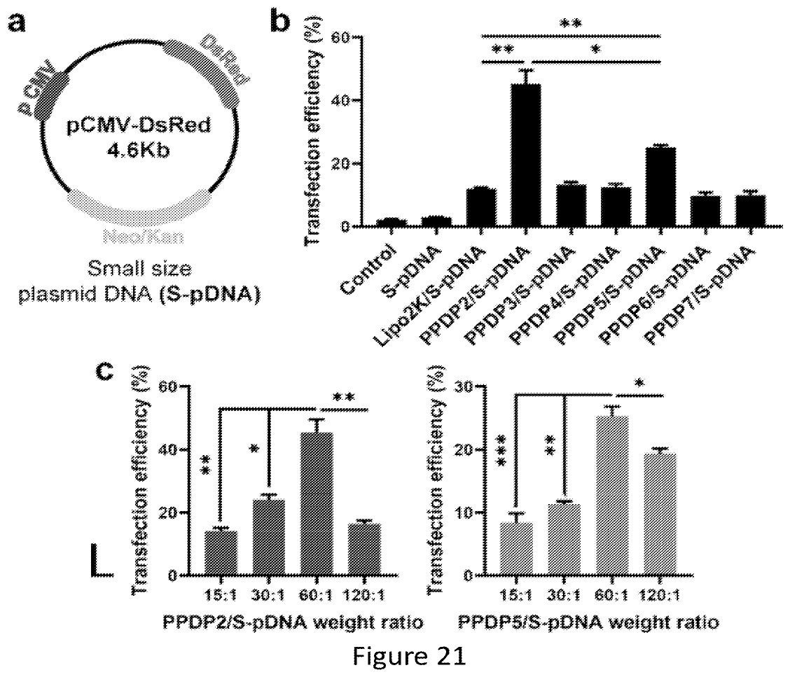

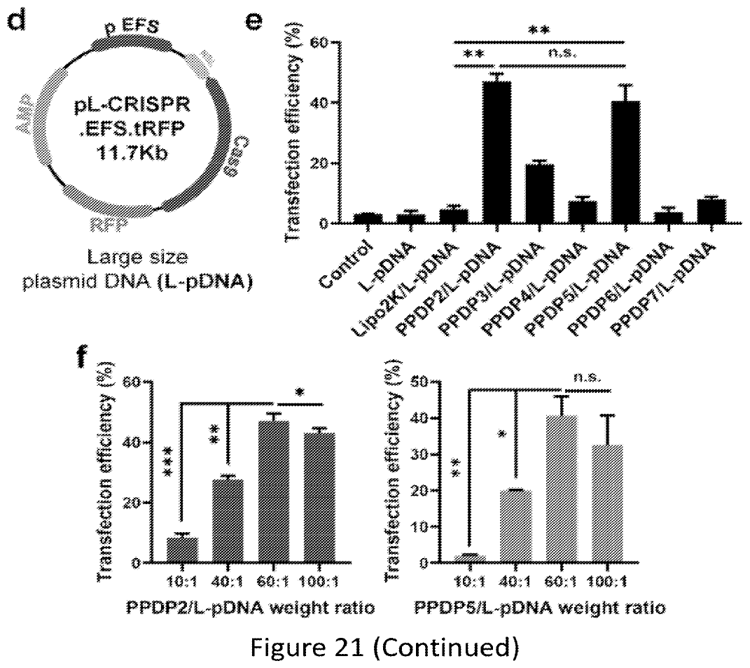

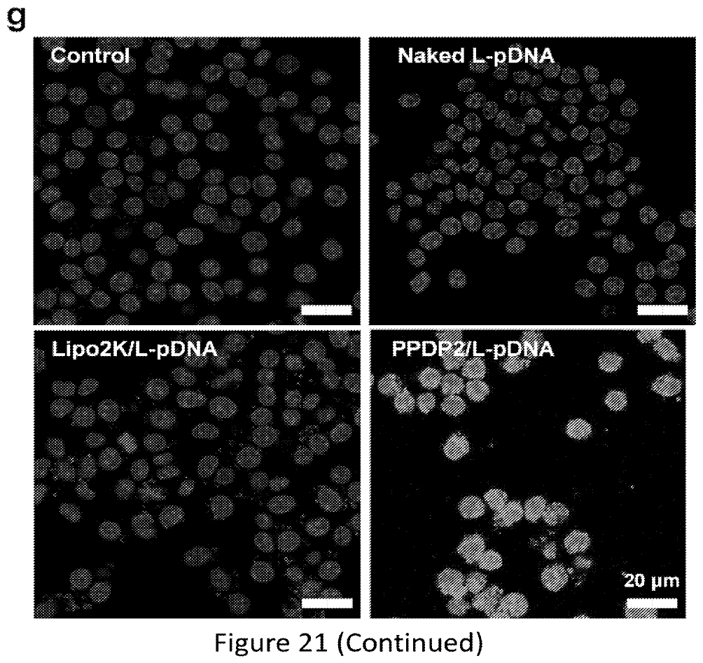

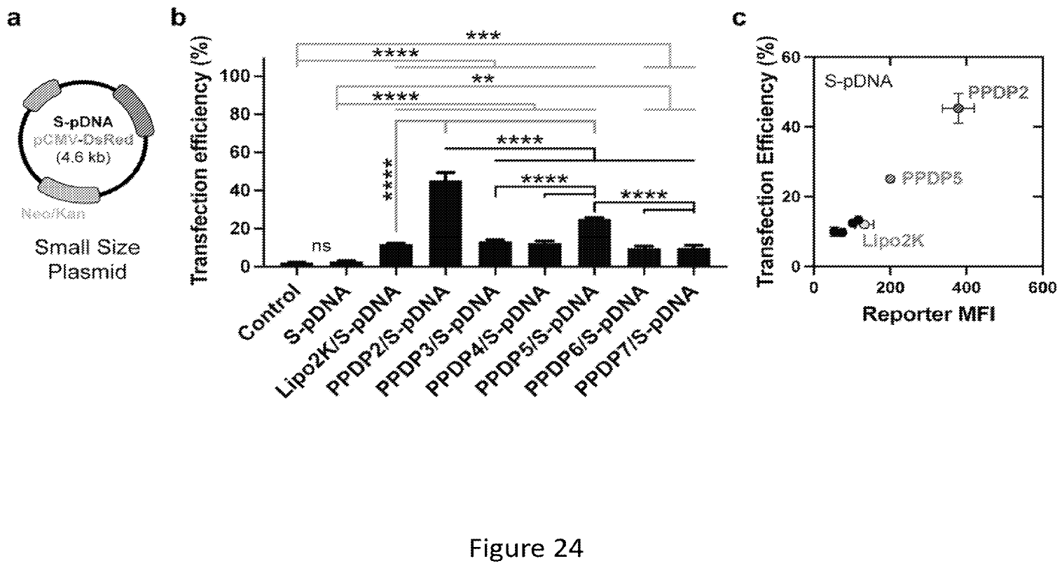

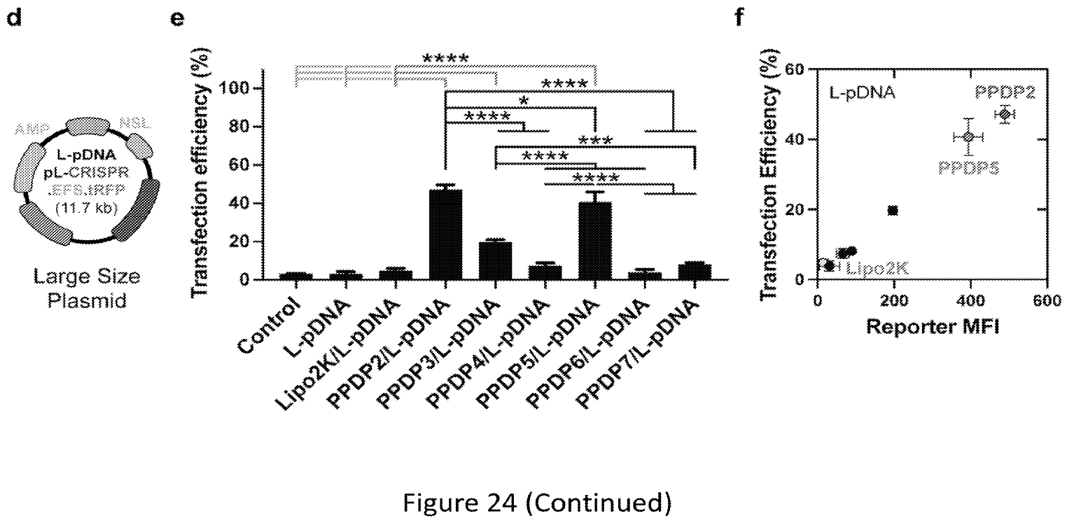

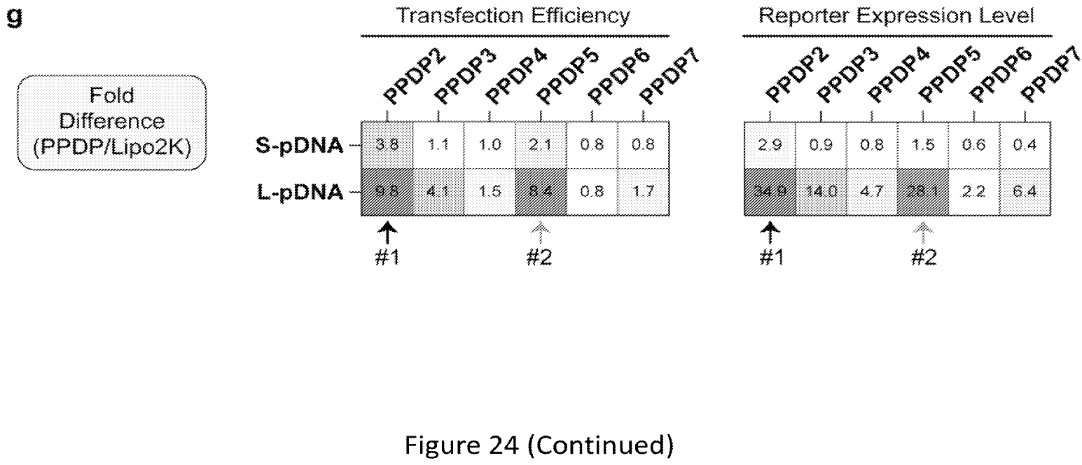

A series of screens in cultured RAW264.7 macrophages to evaluate the performance of each PPDP nanovector as a gene delivery vehicle. Transfection using industry standard Lipo2K following manufacturer recommendations was used to benchmark the performance of the PPDP platform. Furthermore, to understand whether PPDP permits the transfection of pDNA of a diverse size range, screens were performed using plasmids of two different molecular weights. To evaluate the ability of PPDP nanostructures for gene delivery, we used the fluorescence (dsRed or EGFP) driven by a CMV promoter with the size of 4.7 Kb as the small DNA system (FIG. 3A, FIG. 24, FIG. 21a, e.g., S-pDNA (pCMV-DsRed, 4.6 kb) was used as a model small plasmid (FIG. 24, a-c;), while the RFP-tagged CRISPR driven by an EFS promoter with the size of 11.7 Kb as the large DNA system (FIG. 4A, FIG. 21d, L-pDNA (pL-CRISPR.EFS.tRFP, 11.7 kb) was used as a model large plasmid (FIG. 24, d-f), which contains a EFS (elongation factor 1α short) promoter and co-expresses RFP together with the Cas9 protein).

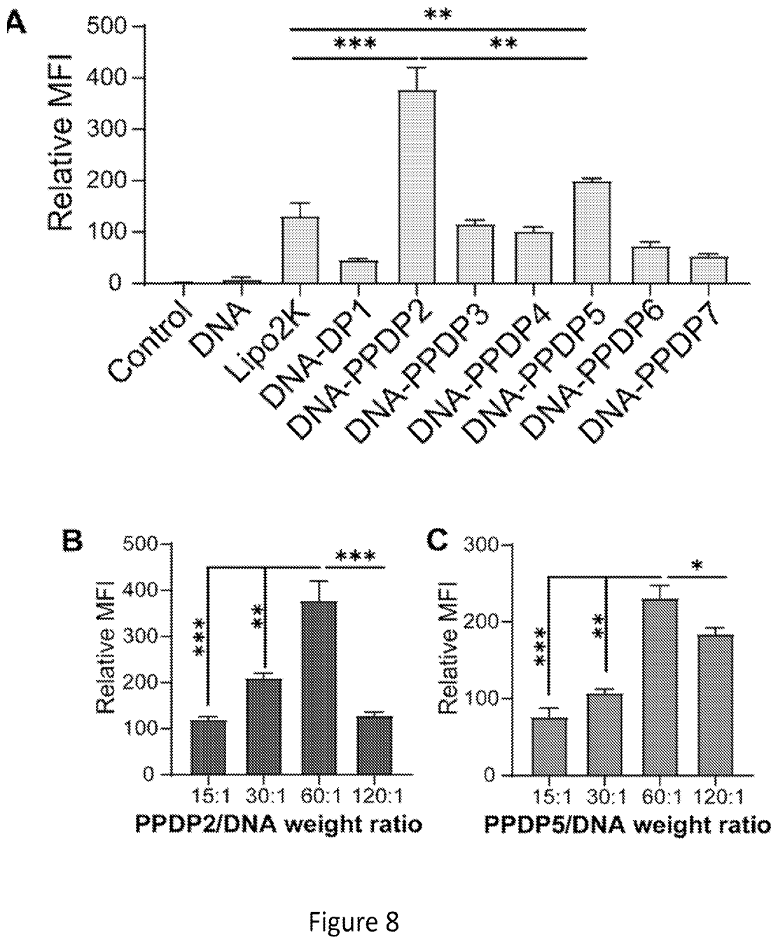

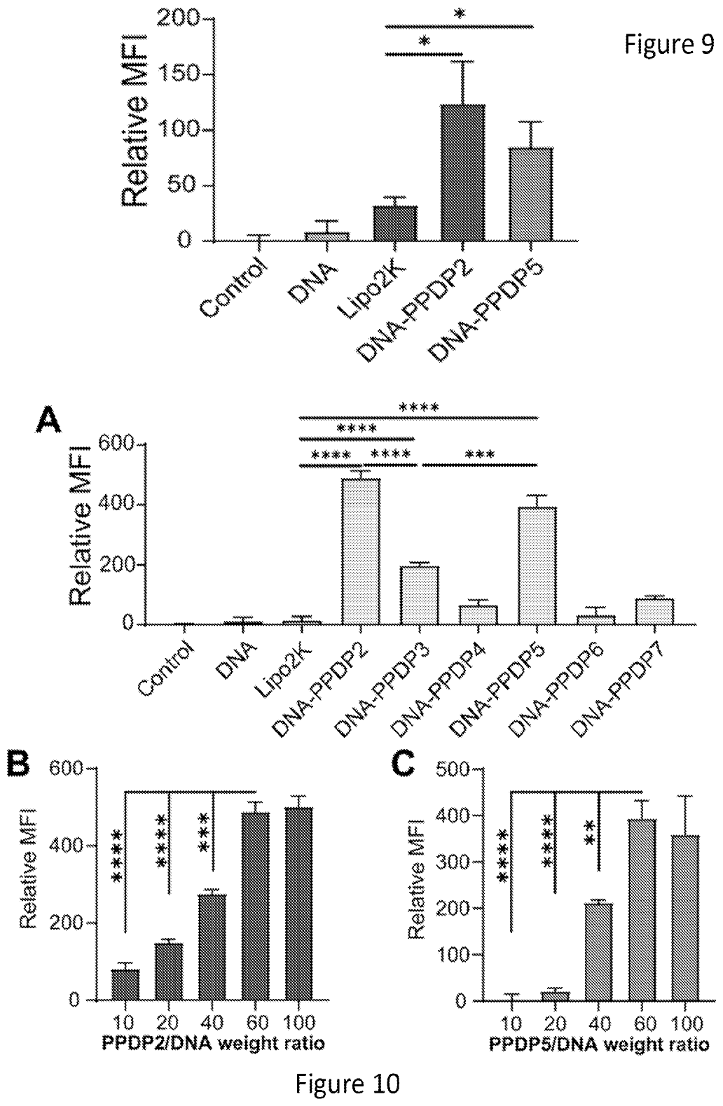

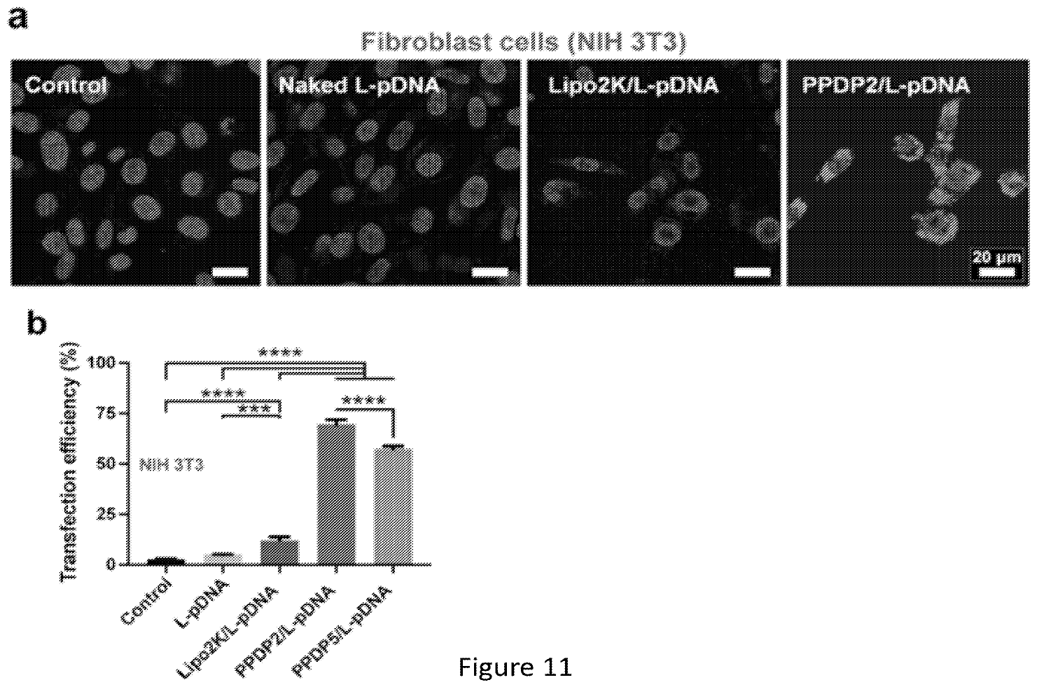

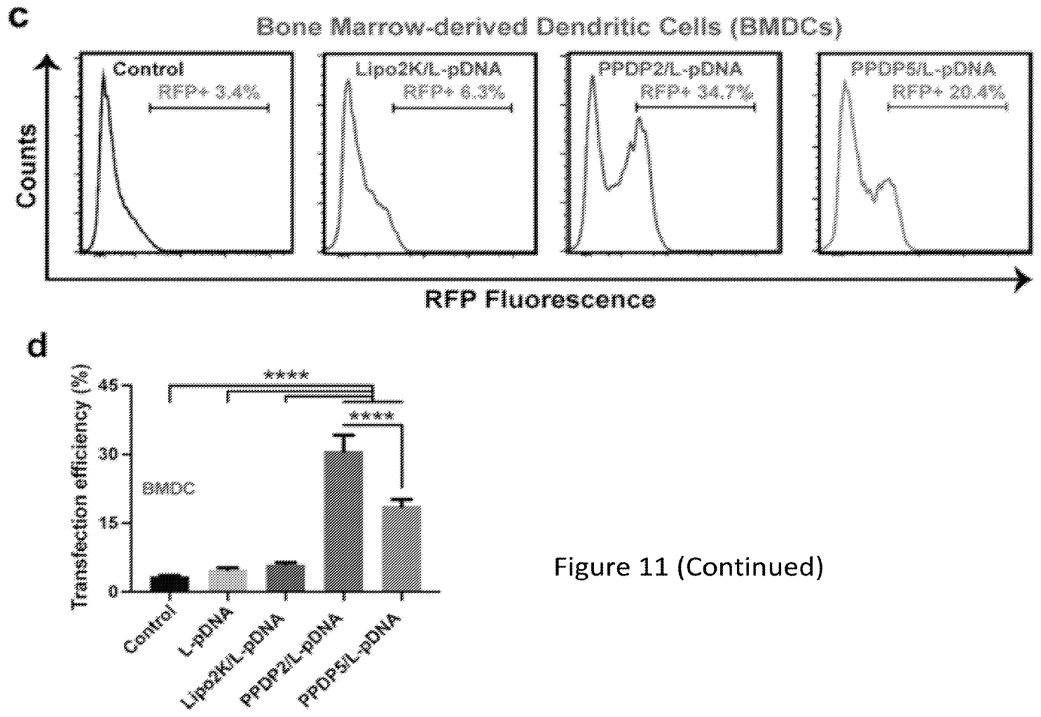

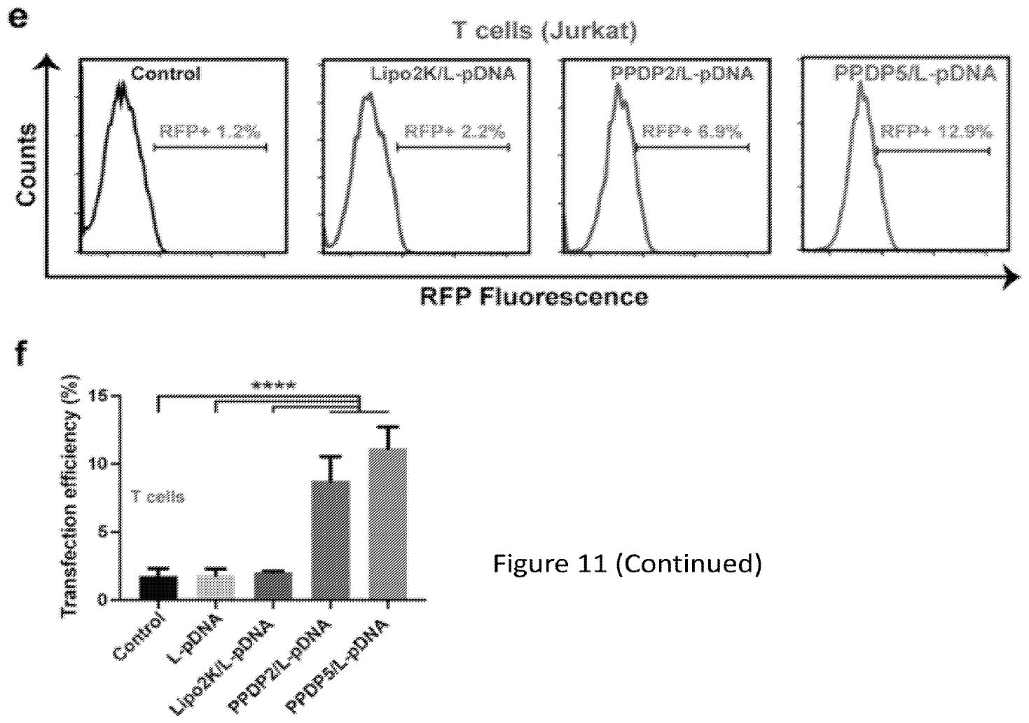

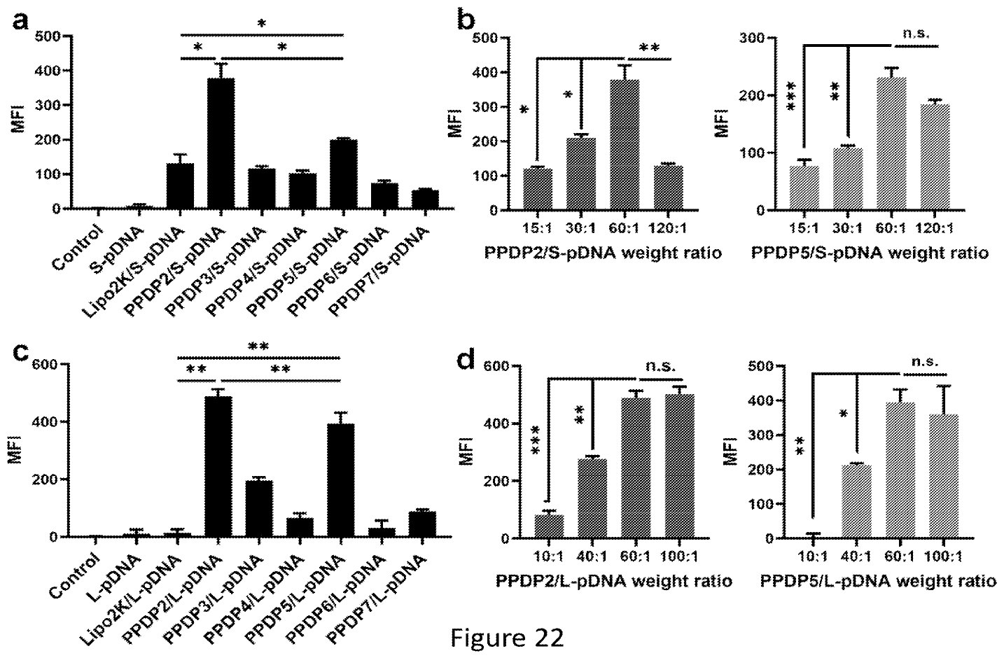

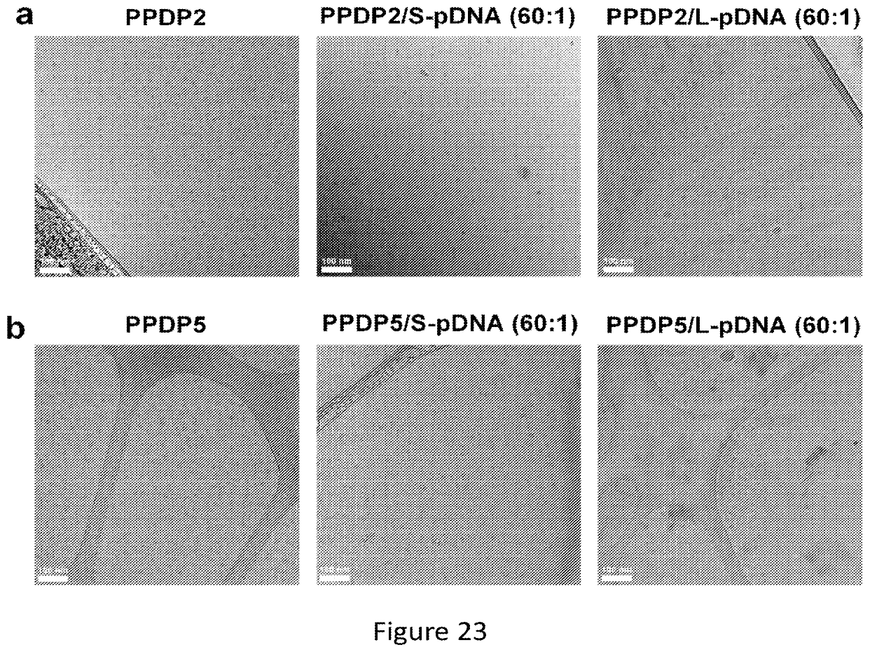

PPDP/pDNA complexes were formed by gently pipetting pre-formed PPDP nanostructures with plasmids followed by 30 min of mixing at room temperature. RAW264.7 macrophages were transfected with pCMV-dsRed (4.6 kb) using a variety of PPDP nanostructures, dendritic peptide control, and Lipo2K control in medium supplemented with serum. After 48 h, the transfection efficiency, including percentages of transfected cells and expression of the fluorescence transgene, was determined using flow cytometry. The expression level of the plasmid-encoded fluorescent protein was also quantified to understand the extent of transfection (FIG. 24c,f;). Gene transfection efficiency increased with increasing PPS content in macrophages. In FIG. 3B and FIG. 8A, the transfection efficiency of pCMV-dsRed (4.6 kb) using PPDP2 was significantly higher than using PPDP5 and any other PPDP nanostructures, with 45.3% of dsRed-positive cells transfected with DNA-PPDP2 versus 25% of dsRed-positive cells transfected with DNA-PPDP5 (p<0.01) (FIG. 24b,). For transfection of pCMV-dsRed (4.6 kb) in macrophages, PPDP2 and PPDP5 were much more efficient than commercial transfection agent Lipo2K (p<0.001). Comparable to the small-sized plasmid, both PPDP2 and PPDP5 indicated their robustness to deliver dramatically larger pEFS-RFP plasmid DNA (11.7 kb), with a high transfection efficiency of 47% and 40% in macrophages respectively (FIG. 4B, FIG. 10A). In particular and strikingly, the transfection efficiency of PPDP2 was ˜10 times higher than Lipo2K (4.8%, p<0.0001). More RFP protein expression in macrophages transfected with pEFS-RFP by PPDP2 compared to Lipo2K can be further confirmed using CLSM (FIG. 4C, FIG. 11A). Given the hydrophobic PPS lengths of PPDP2 (80) and PPDP5 (74) were longer than the other PPDPs, these results support our hypothesis that hydrophobicity played an essential role in gene delivery by PPDP polymers. The optimal DNA-PPDP nanocomplexes composition for pCMV-dsRed (4.6 kb) and pEFS-RFP (11.7 Kb) transfection was the same for both PPDP2 and PPDP5 (at the polymer/DNA weight ratio of 60:1) (FIG. 3C-D, FIG. 8B-C, FIG. 4D-E, FIG. 10B-C, FIG. 22). PPDP2 and PPDP5 with the polymer to DNA weight ratio of 60:1 were therefore selected to verify their gene delivery capability to other cell types (FIG. 23). PPDP2 again performed significantly better than Lipo2K and all other PPDP constructs in these studies using the larger L-pDNA plasmid, achieving significantly greater RFP reporter expression levels.

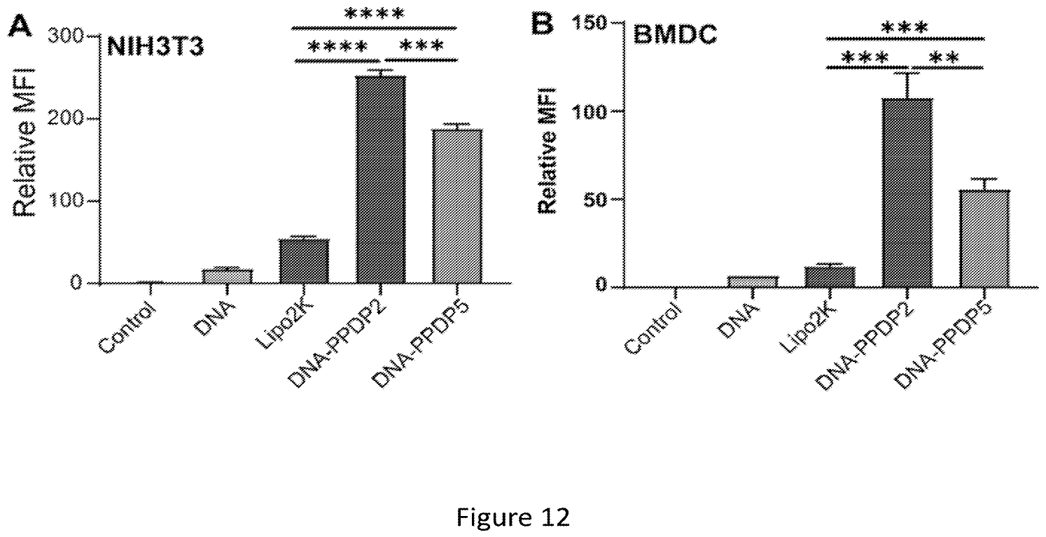

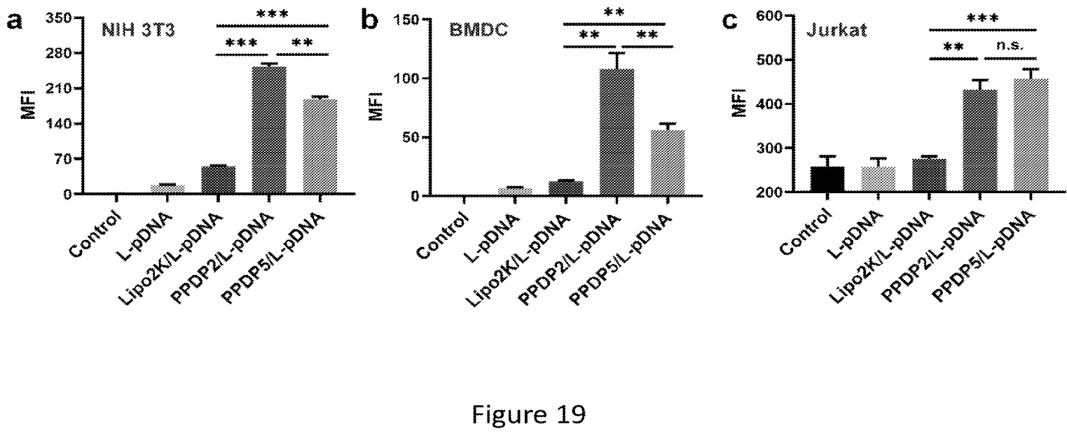

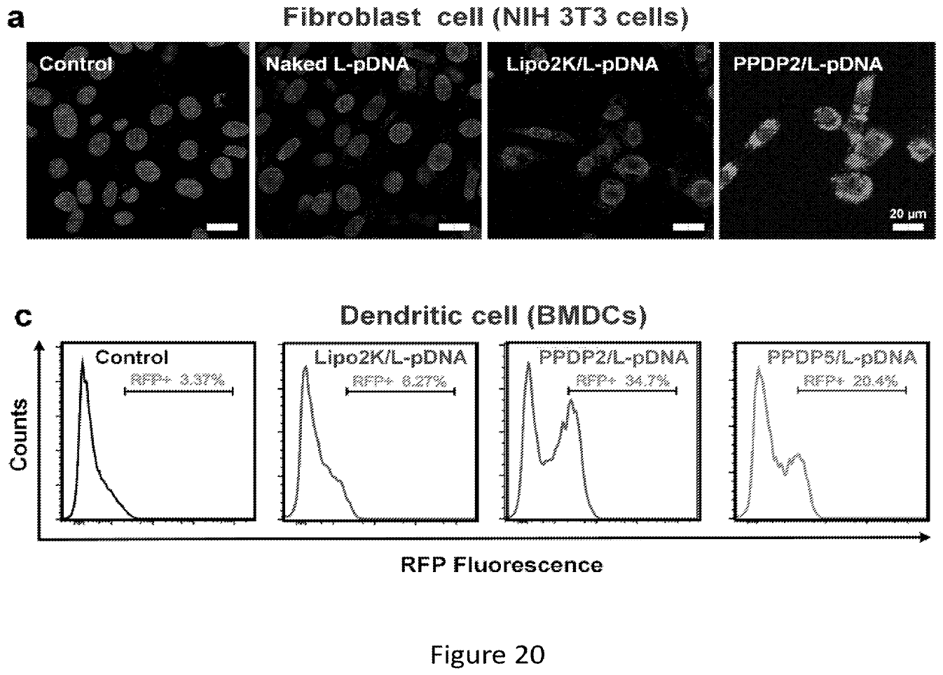

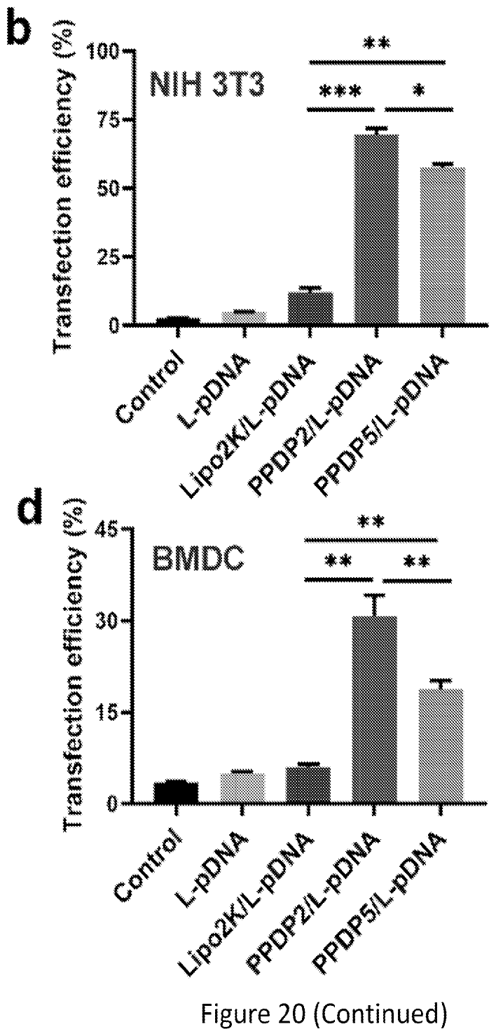

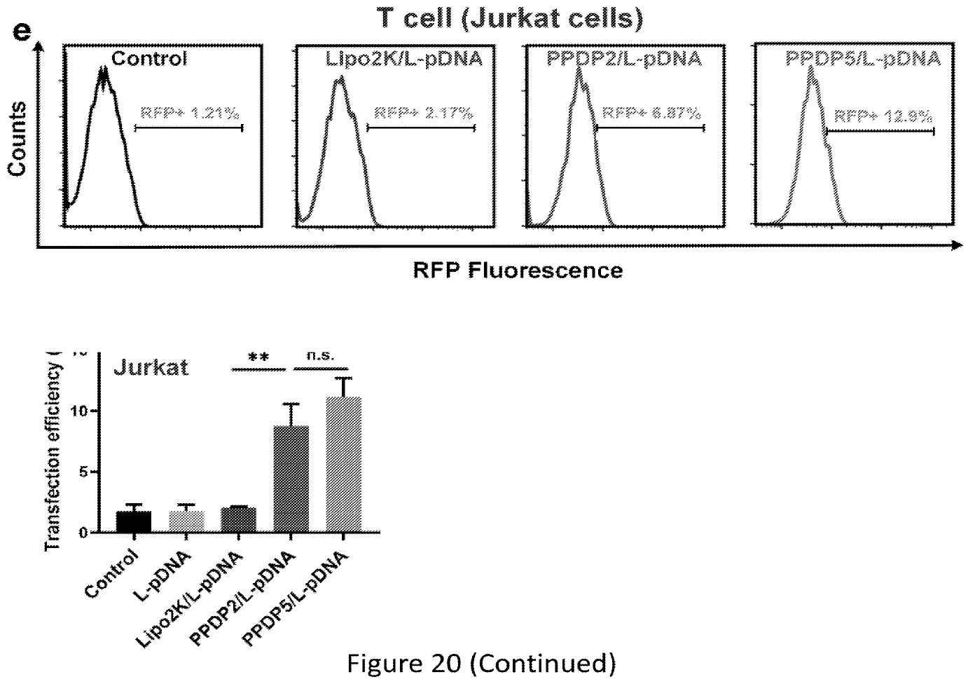

To verify the broad applicability of PPDP2/PPDP5 as potential DNA delivery vehicles, we determined the transfection of plasmid DNA with different sizes in diverse cell lines (FIG. 19, FIG. 20). In NIH3T3 fibroblast cells, PPDP2 and PPDP5 showed 69.6% and 57.5% transfection efficiency, respectively, which were significantly higher than 12% transfection efficiency of Lipo2K (p<0.0001) (FIG. 4F, FIG. 12A). Brighter RFP fluorescence expression was also visible in NIH3T3 cells transfected by DNA-PPDP2 than DNA-Lipo2K after 48 h (FIG. 4G, FIG. 11B).

Due to the unique role of immune regulation, genetic engineered DC has shown as a potentially potent strategy for next-generation vaccines in cancer and infectious diseases. However, progress in DC-based therapies has been obstructed by challenges in genetically manipulating primary DCs. As shown in FIG. 3E-F, the primary cells of mouse bone marrow-derived dendritic cells (BMDCs) were transfected with pCMV-EGFP (4.6 kb) by PPDP2, and PPDP5 resulted in 19.6% and 13.8% of GFP⁺ cells, which were significantly higher than 6.8% of GFP⁺ cells transfected by Lipo2K (p<0.01). Even it's more difficult for large DNA transfection in primary immune cells by the potent commercially available transfection reagent, PPDP2 and PPDP5 could still dramatically improve the delivery and transfection of large plasmid DNA (pEFS-RFP, 11.7 kb), compared to both the naked plasmid DNA (p<0.001) and Lipo2K (p<0.001) (FIG. 4H-I, FIG. 12B). Furthermore, with the same dose of plasmid and polymer, PPDP2 led to 30.7% of RFP positive cells for the large pEFS-RFP delivery, with significantly higher transfection efficiency than PPDP5 (18.7% of RFP*).

### Endosomal Escape and Cytosolic Delivery of DNA

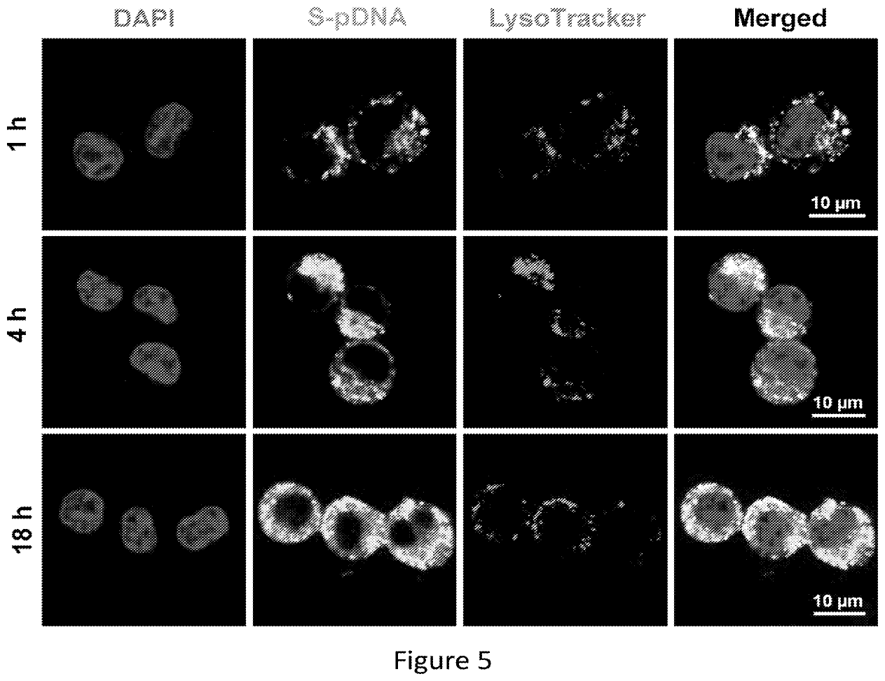

It is noteworthy that successful endosomal escape and cytosolic delivery of DNA is crucial to an efficient gene delivery system. Because of the best transfection capacity among the nanostructures, PPDP2 was chosen to investigate the internalization and localization of the DNA cargo. The intracellular trafficking of Alexa Fluor 488-labeled DNA (488-DNA) PPDP2 nanocomplexes was characterized by visualizing the intracellular distributions of the nanocomplexes at different time points using confocal laser scanning microscopy (CLSM) imaging. As shown in FIG. 5, after 1 h incubation with macrophages, the 488-DNA fluorescence (green) could be observed in the cytosol, with some level of colocalization with endosome/lysosome (yellow), suggesting the efficient endosome escape of 488-DNA-PPDP2 nanocomplexes. With longer incubation time (4 h and 18 h), more nanocomplexes were released into the cytosol, and some were still entrapped inside and endosomal/lysosomal vesicles, indicating the nanocomplexes escaping from endosome were time-dependent.

The PPDP Platform is Less Cytotoxic than Commercial Lipo2K and Polyethylenimine (PEI) Reagents

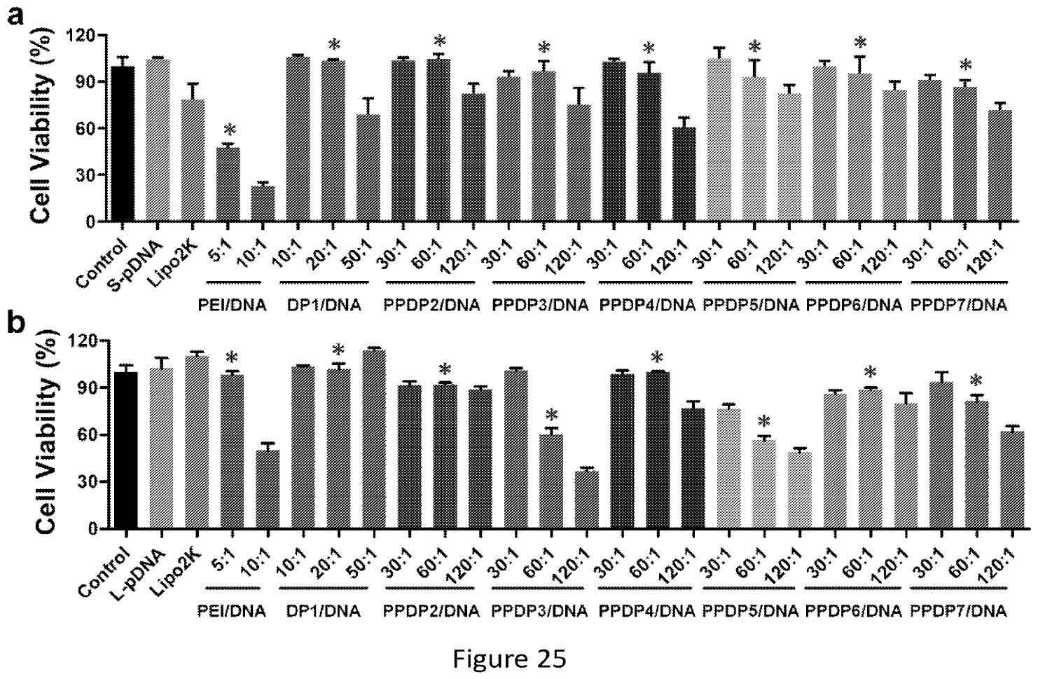

Cytotoxicity of PPDP/pDNA nanocomplexes prepared at various weight ratios was examined in RAW264.7 macrophage cells (FIG. 25, a-b), which. The cytotoxicity of PPDP/pDNA nanocomplexes was examined using the 3-(4,5-dimethylthiazol-2-yl)-2,5-diphenyltetrazolium bromide (MTT) assay. These toxicity studies were benchmarked against both Lipo2K, which is the leading commercially available transfection reagent, and the cationic polyethylenimine (PEI) polymer. PEI (25 kDa) was included as a control, since it is a widely used polymeric transfection reagent that has known toxicity concems^{[28-30]}.

PPDP/pDNA nanocomplexes were generally non-toxic (cell viability >80%) under weight ratio of 60:1 (PPDP: both S-pDNA and L-pDNA), whereas cell viability was lower for nanocomplexes having a PPDP/pDNA ratio of 120:1 (FIG. 25, a-b). However, decreases in cell viability were also observed for the unconjugated DP peptide, even at a peptide/S-pDNA weight ratio less than 60:1. These results suggest that conjugating DP to PEG-b-PPS copolymer significantly reduces toxicity of the peptide. Importantly, the cytotoxicity of PPDP/pDNA nanocomplexes was much lower than the commercial polymeric transfection agent PEI (FIG. 25, a-b). High (˜90-100%) cell viability was observed for PPDP2 up to the 60:1 polymer-to-pDNA ratio for both large and small plasmids, which was generally less cytotoxic than PPDP5 (FIG. 25, a-b).

Collectively, the results from our screening studies demonstrate that PPDP2 and PPDP5 are technologies that outperform the industrial standard transfection reagents. Given the longer hydrophobic PPS lengths of PPDP2 (80 units) and PPDP5 (74 units) compared to the other more toxic PPDP polymer variants, the increased hydrophobicity may play a role in reducing toxicity, possibly by modulating the structure and/or stability of the complexes. The emergence of PPDP2 and PPDP5 from these initial screening efforts led us to focus subsequent development efforts around these prototypes. The proceeding work emphasized the PPDP2 nanovector, since it outperformed all other prototypes by a large margin, including PPDP5 (FIG. 24c,f,g). Furthermore, PPDP2 performed at a higher level while exhibiting even lower cytotoxicity than PPDP5 (FIG. 25).

Optimization of PPDP Polymer for Plasmid DNA Binding Capability-PPDP/pDNA Nanocomplexes Formed at a 60:1 Polymer-to-Plasmid Ratio Optimally Bind to pDNA and Form Stable Nanostructures

To investigate the ability of the polymers to bind and condense plasmid DNA (pDNA), we next examined the formation and stability of PPDP/pDNA nanocomplexes using an electrophoretic mobility shift assay (EMSA). In this assay, the DNA will remain in the well of the gel if the PPDP/pDNA nanocomplex is stable once an electric field is applied to commence electrophoresis. On the other hand, if the nanocomplex is unstable, the unbound pDNA will migrate down the agarose gel toward the positive electrode in the presence of the electric field. These studies employed both small (S-pDNA, 4.6 kb) and large (L-pDNA, 11.7 kb) plasmids to understand whether or not different plasmid sizes required different polymer-to-plasmid ratios to perform optimally. The PPDP/pDNA complexes were formulated by combining PPDP with pDNA at various polymer-to-pDNA weight ratios (PPDP:pDNA=1:1-120:1) for EMSA experiments (FIG. 15, a-b).

The migration of pDNA was completely obstructed by PPDP nanostructures having a polymer/pDNA weight ratio greater than 15:1 (FIG. 15, a-b). Notably, the accessibility of pDNA to the GelRed® nucleic acid stain progressively decreased with increasing polymer/pDNA ratio, suggesting enhanced pDNA binding for a more compact nanostructure in response to increased relative amounts of copolymer (FIG. 15, a-b). These results verify that PPDP polymer is capable of binding to S-pDNA (FIG. 15a) and L-pDNA (FIG. 15b). Of note, PPDP2/pDNA and PPDP5/pDNA showed the lowest pDNA fluorescent intensity at weight ratio of 60:1 (PPDP: both S-pDNA and L-pDNA) in the wells, indicating an optimal pDNA binding capability.

Next, we examined the effect of the mass ratio of PPDP polymer to pDNA on the particle size and zeta potential (FIG. 15, c-d). As this ratio increased, the size of the nanocomplex increased and the zeta potential became more positive (FIG. 4, c-d). Nanocomplexes ranged from negative, to neutral, to positively charged as the PPDP/pDNA ratio was increased from 1:1 to 60:1. Based on the EMSA results (FIG. 15, a-b), size and zeta potential characterization (FIG. 15, c-d), and cytotoxicity studies (FIG. 25), we selected the optimal ratio of 60:1 for further gene transfection studies. When containing either S-pDNA or L-pDNA, 60:1 PPDP/pDNA nanocomplexes were observed to be spherical and monodisperse nanostructures, as observed by cryogenic transmission electron microscopy (cryoTEM) (FIG. 15, e-f;).

The diameter and zeta potential of the nanocarriers described herein can have a wide range of zeta potentials and diameters. For example, in some embodiments, the nanocarrier has a zeta potential between +80 to −80 and diameters between 5 nm-500 nm.

The PPDP system uses both self-assembly and electrostatic complexation simultaneously. A high polymer to payload ratio is standard for self-assembling systems, but high ratios are required for DNA/polymer complexes. PPDP is between these two, as it requires a higher amount of polymer so that the PPDP influences the assembly of the complexes, which may explain the uniformity of the nanostructures (FIG. 15).

The 60:1 PPDP:pDNA Ratio Achieves Optimal Transfection Efficiencies In Vitro

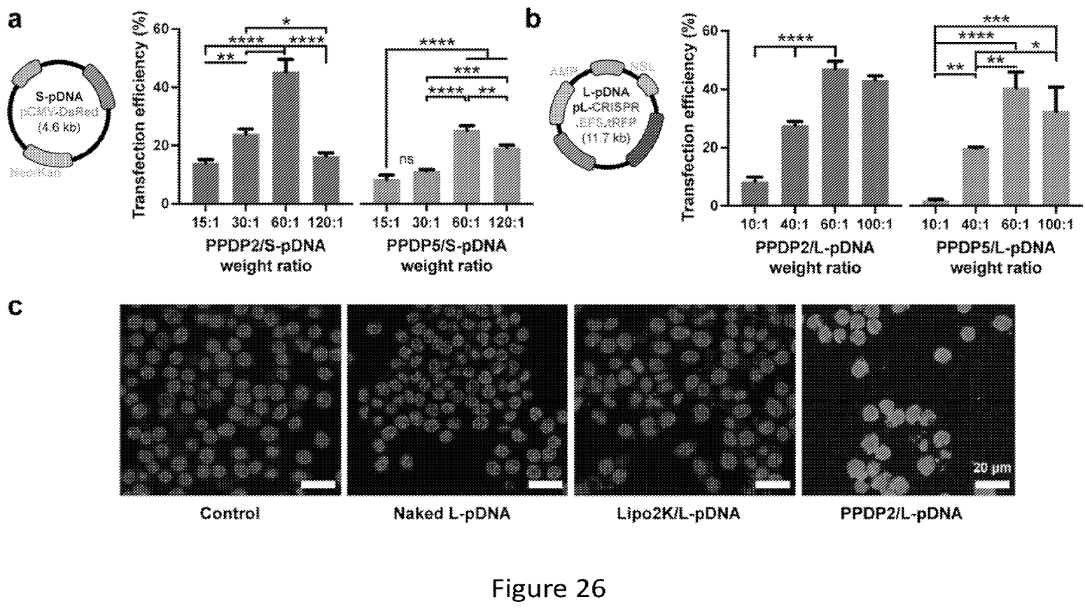

We next examined the relationship between the PPDP:pDNA ratio and cellular transfection performance using the highest performing PPDP2 and PPDP5 constructs. We hypothesized that the 60:1 PPDP:pDNA ratio would also perform optimally in these studies as it demonstrated the most stable binding to plasmid in the EMSA studies. Furthermore, we also examined whether less economical PPDP:pDNA ratios exceeding 60:1 would provide any performance benefits at the cost of requiring additional copolymer in the formulation. Macrophages were transfected with PPDP:pDNA complexes, and the transfection efficiency (FIG. 26, a-b) and expression level of the plasmid-encoded reporter proteins was quantified by flow cytometry.

For both PPDP2 and PPDP5, the highest transfection efficiencies were observed for nanovectors prepared at the 60:1 PPDP:pDNA ratio for both small (FIG. 26a) and large (FIG. 26b) model plasmids. With exception to the PPDP2/L-pDNA results, the 60:1 ratio performed significantly better than all other PPDP:pDNA ratios tested (FIG. 26, a-b). In the case of the exception, we note that the PPDP2-mediated transfection of the large L-pDNA plasmid was more efficient at a 60:1 ratio than a higher 100:1 ratio, although this difference was not statistically significant (FIG. 26b). Nevertheless, these results demonstrate that the 60:1 PPDP:pDNA ratio is optimal for transfection with both small and large plasmids, and there is no benefit to increasing the amount of polymer used relative to the pDNA. Confocal laser scanning microscopy (CLSM) further confirmed that the level of RFP protein expression in macrophages transfected with L-pDNA using PPDP2 was greater than the RFP expression level achieved by Lipo2K (FIG. 26c).

PPDP undergoes a disorder-to-order transition into a unique helical conformation under acidic conditions and promotes the intracellular release of pDNA cargo—The dendritic peptide conjugate of PPDP nanovectors adopt a unique helical conformation under pH 6.0

The results of our preliminary screening and polymer-to-DNA ratio optimization studies demonstrated that PPDP2 prepared at a 60:1 PPDP:pDNA consistently outperformed all other nanovectors and commercial reagents. Thus, this PPDP2 formulation was investigated further in mechanistic studies seeking to characterize its endolysosomal escape properties. Based on the biochemical properties of the DP amino acid composition (FIG. 16a), we hypothesized that the conformation of this peptide would change in increasingly acidic environments.

To assess the pH-responsive structure of PPDP, spherical nanostructures were self-assembled from PPDP2 copolymer (FIG. 16b), which demonstrated a mean diameter of 19.6 nm and surface charge of 48.0 mV as measured by DLS (FIG. 16c). The DP component of the PPDP2 nanostructures is readily detectable by Fourier transform infrared (FTIR) spectroscopy in solution (FIG. 16, d-e). PPDP2 nanostructures exhibited the expected amide I peak in the 1700-1600 cm⁻¹ band (C═O stretching), which is characteristic of peptide bonds (FIG. 6d). As expected, this peak was also detectable in the DP peptide control, although its intensity was partially masked by a stronger peak in the 1800-1700 cm⁻¹ band. Subtracting the PEG-b-PPS spectra from that of the PPDP2 sample enables a preliminary assessment of the helical secondary structure. In the difference spectra presented in FIG. 6e, a strong peak is observed near 1660 cm⁻¹ within the amide I band. These results suggested that PPDP2 may adopt a helical secondary structure.

We next performed circular dichroism (CD) spectroscopy to more thoroughly examine if the dendritic peptide adopts a helical secondary structure and whether a conformational change occurs in response to a shift from pH 7.5 to pH 5.5 (FIG. 16, f-g). This pH range is particularly interesting in the context of a nanostructure trafficking through the endolysosomal pathway that becomes progressively more acidic. In our biophysical studies, PPDP2 did not take on a traditional alpha-helix secondary structure under any condition, which would be indicated by two negative peaks at 208 nm and 222 nm in a CD spectrum^{[57]} (FIG. 16f).

We initially suspected that PPDP2 was intrinsically disordered, which is a ubiquitous feature of many peptides and proteins^{[58-67]}. Closer inspection of the PPDP2 CD spectra instead provides evidence of a different, more esoteric, type of helical structure resembling that of PPII (polyproline 11)-like structures, which can be formed by peptides lacking proline residues^{[68]} and have a characteristic combination of spectral features of disordered and ordered states (FIG. 16f). Proteins and peptides adopting a random coil state commonly have a negative peak near 200 nm. However, a PPII-like helical structure is revealed by a characteristic positive peak near 220 nm^{[69]} (but as low as 210 nm for proline-lacking peptides adopting a PPII-like conformation^{[70]}) and minima at 197 nm^{[70]}, which is a spectral signature that is used to distinguish these special helical conformations from purely disordered states. In our studies, PPDP2 exhibited a strong negative peak at ˜201 nm at pH 7.5 and at ˜204 nm at pH 6.5, whereas the most acidic conditions (pH 5.5) induced a strong leftward shift to a minimum at ˜197 nm. The key feature that distinguishes the PPDP2 CD spectra from a disordered spectrum is observed at pH 5.5, where a strong positive peak is observed at 215 nm (FIG. 16f). Thus, the PPDP2 CD spectrum contains both a negative peak at 197 nm and a positive peak at 215 nm under conditions that mimic a more strongly acidic intracellular environment, suggesting it adopts a PPII-like helical conformation below pH 6.5. This interpretation is consistent with past studies that demonstrate lysine homopolymers adopt a PPII-like helical structures with similar spectral features^{[71-73]}, which can be stabilized by either the conformation of lysine in the backbone^{[71]0} or by electrostatic interactions^{[74]}. Since the lysine side chains within the dendritic peptide are employed for branching (FIG. 1) and thus not available for electrostatic interactions, the chemical bonding of lysine residues within the dendritic peptide backbone likely promotes the formation of a PPII-like helical conformation.

Interestingly, the free dendritic peptide (unconjugated control) exhibits a peak at ˜195 nm at pH 7.5 and pH 6.5, and a peak at ˜200 nm at pH 5.5 (FIG. 16g). This unconjugated dendritic peptide also exhibits the positive peak (˜215 nm) at all pH values, suggesting it has the PPII-like helical structure under all conditions. Since the positive peak is only observed a pH 5.5 for PPDP2, these results suggest that conjugating the peptide to the polymer rendered the peptide conformation pH-responsive. In the context of a gene delivery technology, the absence of the ˜215 nm positive peak at pH 7.5 and pH 6.5 in the PPDP2 CD spectra suggest the peptide is disordered prior to reaching acidic lysosomal compartment. Yet once PPDP2 reaches the lysosome (pH<6), it can adopt a helical conformation. This PPDP2 disorder-to-order transition that occurs in acidic environments may provide a mechanism for lysosomal escape. At all pH values examined, the ionizable guanidino group of arginine (pKa ˜9) in PPDP2 is protonated and provides a stable electrostatic interaction with DNA. At a pH below 6.0, the imidazole group (pKa ˜6) of histidine residues should be protonated and provide an additional means for electrostatic interaction with DNA. Stabilizing the PPDP2-DNA interaction within acid lysosomal compartments is important, as the higher order nanostructure disassembles under these conditions due to oxidation of PPSE^{[42]}. Furthermore, the protonated histidine residues are also known to facilitate membrane disruption in concert with leucine residues that play a role in membrane binding^{[56,75]}. Consequently, the oxidation-mediated loss in PPS hydrophobicity as well as the enhancement in DNA-binding and membrane-disruption capabilities of the dendritic peptide under acidic conditions, all coincide with the adoption of a PPII-like helix within the lysosome. Of note, helical conformations are known to promote the cytoplasmic release of cargo from lysosomal compartments^{[76,77]}. Thus, we conclude that the formation of helical structures acts synergistically with the functionality of the ionized amino acid residues and the oxidized PPS moieties of PPDP2 at low pH (pH<6) to promote the efficient lysosomal release of DNA cargo into the cytoplasm.

PPDP Traffics Through the Endolysosomal Pathway and Releases pDNA Payloads into the Cytoplasm in a Time-Dependent Fashion

We sought to investigate the cellular internalization and localization of DNA cargo delivered using the pH-responsive PPDP2 nanovector. To this end, CLSM imaging was used to visualize the intracellular trafficking of PPDP2/Alexa Fluor 488-labelled pDNA (pcDNA3.1, 5.4 kb) nanocomplexes at different time points. Early 1 h and 4 h timepoints were included to examine nanovector migration through the endolysosomal pathway, as well as the onset of endosomal escape, whereas an 18 h timepoint was included to examine changes in the cytoplasmic accumulation of the pDNA payload with time (FIG. 5). After 1 h incubation with RAW264.7 macrophages, the green fluorescence (Alexa Fluor 488) was predominantly observed as puncta that co-localized with endosomal/lysosomal compartments. At longer incubation times of 4 h and 18 h, nanocomplexes escaped from endolysosomal compartments and were released into the cytosol. Cytoplasmic release is observed by the presence of diffuse green fluorescence that no longer colocalizes with the endosomal/lysosomal compartments. A fraction of the nanocomplexes were still entrapped within endosomal/lysosomal compartments at the 4 h and 18 h timepoints. However, the diffuse green signal was much greater in intensity at 18 h compared to 4 h, and this change also coincided with a decrease in the co-localization signal at 18 h. These results demonstrate that Alexa Fluor 488-labelled pDNA efficiently and increasingly escaped from the lysosome over time following intracellular delivery via PPDP2 nanocomplexes.

### The PPDP Platform Efficiently Transfects Both Innate and Adaptive Immune Cells

To verify the broad applicability of PPDP2 and PPDP5 as potential DNA delivery vectors, we assessed L-pDNA transfection of diverse cell lines (FIG. 11). Transfection was performed under the standard culture conditions for each cell type in the presence of serum. We first tested NIH 3T3 mouse fibroblast cells, which have been widely used for DNA transfection studies and recombinant protein expression in biological research. Confocal imaging revealed extensive RFP fluorescence expression after 48 h in NIH 3T3 cells that were transfected by PPDP2/L-pDNA, but minimal to no detectable signal for Naked L-pDNA and Lipo2K/L-pDNA at the same timepoint (FIG. 11a). Flow cytometry analysis demonstrated that PPDP2 and PPDP5 exhibited transfection efficiencies of 69.6% and 57.5%, respectively, in fibroblasts (FIG. 11b). These transfection efficiencies were significantly greater than the 12% transfection efficiency determined for Lipo2K (FIG. 11b). Consistent with our results in macrophages, PPDP2 transfected fibroblasts with an efficiency that was significantly greater than that of PPDP5 (FIG. 11b).

We next examined whether the PPDP technologies could efficiently transfect dendritic cells (DCs). Developing technologies to facilitate the production of genetically engineered DCs is of great interest due to their unique and versatile role in immune regulation and their potential use in creating next-generation vaccines for cancer and infectious disease. However, the genetic manipulation of primary DCs remains challenging and continues to obstruct progress in developing DC-based therapies. Primary mouse bone marrow-derived dendritic cells (BMDCs) were transfected with L-pDNA using PPDP2 and PPDP5 (FIG. 11, c-d). PPDP2-mediated transfection resulted in 30.7% RFP positive (RFP+) cells, whereas 18.7% of cells were RFP+ after transfection with PPDP5 (FIG. 11, c-d). These percentages were significantly greater than both naked plasmid DNA and Lipo2K controls (FIG. 11, c-d).

Lastly, we investigated the use of PPDP2 and PPDP5 for transfecting Jurkat T cells with a large plasmid (FIG. 11, e-f). Jurkat T cells are immortalized human T lymphocytes that are commonly employed to study T cell biology and for developing prototypes of engineered T cell technologies. PPDP2 and PPDP5 increased the percentage of RFP+ T cells, as observed by the rightward shift in flow cytometry histograms compared to the negative control group (FIG. 8e). Interestingly, both PPDP2 and PPDP5 achieved transfection efficiencies that were significantly greater than that achieved by Lipo2K (FIG. 11f). While PPDP5 transfected T cells with large plasmids slightly more efficiently than PPDP2, this difference in transfection was not significantly different (FIG. 11f). This trend observed for T cells (FIG. 11f) differs from our observations using fibroblasts and DCs, where PPDP2 achieved transfection efficiencies that were significantly greater than PPDP5 (FIG. 11, b-d). It is unclear why PPDP2 was not also superior for transfecting T cells with large plasmids, however, understanding the mechanism for this difference is outside the scope of the present work. Collectively, these results demonstrate that PPDP2 and PPDP5 dramatically improve the delivery of large plasmid DNA molecules to primary immune cells and achieve greater transfection efficiencies than commercially available transfection reagents (FIG. 11).

Summary of Example 1 Results

We have developed and optimized a polymeric nanovector as a non-viral and nontoxic plasmid transfection reagent for diverse immune cell types without the need for specialized culture conditions or medium. The PPDP vehicle consists of a self-assembling PEG-b-PPS copolymer conjugated to a cationic dendritic peptide. Each branch of the dendritic peptide possesses an arginine terminus for stable complexation with DNA via electrostatic interactions, and the lysine-branched backbone undergoes a helical conformational change in acidic environments that may assist with endosomal escape. This lysosomal escape is a prerequisite for pDNA diffusion into the nucleus where it can be transcribed and translated into a protein product and is thus an essential feature of any effective gene delivery technology and transfection reagent. Nanocarriers assembled from PEG-b-PPS copolymers have previously demonstrated enhanced cytosolic delivery of diverse therapeutic payloads^{[40,43,52]}, and here the conjugation of a cationic dendritic peptide achieves this capability for both small (S-pDNA; 4.6 kb) and large (L-pDNA; 11.7 kb). Screening the size and surface character of PPDP yielded the PPDP2 and PPDP5 constructs, which were found to be optimal for enhanced transfection with significantly less toxicity than the commercial standard lipofectamine in studies using macrophages, fibroblasts, primary BMDCs, and T cells in vitro. While PPDP2 was found to be the more efficient overall, both constructs were particularly useful for transfecting cells with large pDNA elements. PPDP is therefore a promising non-viral vector with numerous advantages for efficient in vitro transfection, including exceptionally low toxicity, proficient cytosolic delivery of large genetic elements, and efficacy under standard culture conditions for typically difficult to transfect immune cell populations.

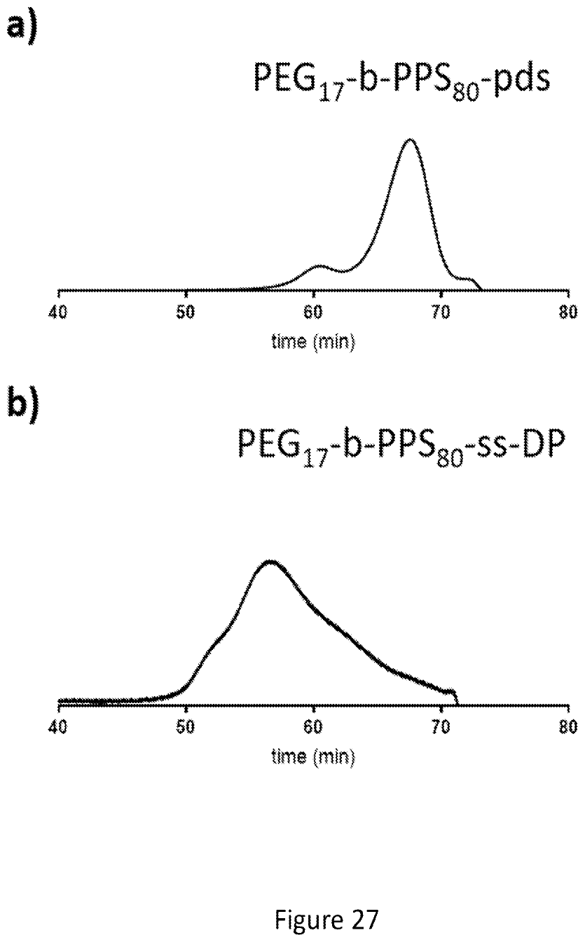

Further, FIG. 27 demonstrates that the polymer conjugation is expected. The data shows the before and after of the conjugation of our PEG-PPS polymer to the dendritic peptide, resulting in a shift in the elution time within a size exclusion chromatography (SEC) column. This verifies the formation of the expected polymer.

Experimental Section/Methods

Materials and cell lines: All chemicals were purchased from Sigma-Aldrich, unless otherwise noted. A dendritic peptide (DP) with the sequence of {[(RHL)₂-KRHL]₂-KRHL}, 2-KC—NH2 was purchased from Peptide 2.0. The murine macrophages RAW 264.7, mouse fibroblasts NIH 3T3, and human Jurkat T cells were purchased from the American Type Culture Collection (ATCC, Inc.). For cell culture, DMEM and RPMI 1640 media, penicillin/streptomycin antibiotics, and fetal bovine serum (FBS) were purchased from Life Technologies. All cell culture medium was supplemented with 10% (v/v) fetal bovine serum (FBS), penicillin (100 IU/mL) and streptomycin (100 pg/mL) at 37° C. with 5% CO2.

Plasmids: Small plasmid DNA: pCMV-DsRed (4.6 kb) was purchased from Clontech Laboratories, pcDNA3.1 (5.4 kb) without a fluorescence tag was purchased from Thermo Fisher Scientific. Large plasmid DNA (L-pDNA): pL-CRISPR.EFS.tRFP (11.7 kb) was purchased from Addgene. All plasmids were propagated in DH5α competent cells (Thermo Fisher Scientific). The plasmid DNA concentration was determined using a NanoDrop 2000 instrument (Thermo Fisher Scientific) by measuring the absorbance at 260 nm.

Generation of mouse primary bone-marrow-derived dendritic cells: Bone-marrow-derived dendritic cells (BMDCs) were prepared as described previously^{[78]}. Briefly, bone marrow cells were collected from the tibias and femurs of naïve C57BL/6 mice (Jackson Laboratory). The cells were then resuspended in primary media (RPMI 1640 medium supplemented with 10% fetal bovine serum (FBS), penicillin (100 IU/mL) and streptomycin (100 mg/mL), B-Me (50 Um), L-Gln (2×10⁻³ m), GM-CSF (20 ng/mL), and IL-4 (10 ng/mL)). Cells were cultured in 100 mm Petri dishes with a density of 1×10⁶ cells/mL and incubated at 37° C. with 5% CO2 for seven days. After 7 days, non-adherent and loosely adherent cells (imDCs) were harvested, washed, and used for in vitro experiments.

Synthesis of PEG-b-PPS-s-s-DP (PPDP) polymers: Block copolymers PEGₘ-b-PPSₙ were synthesized as previously described^{[39,43,79,80]}. Briefly, the poly(ethylene glycol) (PEG)-based initiator was prepared by thioacetate modification of methoxy PEG-OH (MW=750 or 2000). PEG thioacetate initiators were deprotected by sodium methoxide to reveal the initiating thiolate. The amount of propylene sulfide (PPS) used in the reaction was adjusted to polymerize the desired block lengths. The polymerization was end-capped by excess 2,2′-dithiodipyridine (5 equiv). The obtained block copolymers (PEG₁₇-b-PPS₈₀-pds, PEG₁₇-b-PPS₅₁-pds, PEG₁₇-b-PPS₄₂-pds, PEG₄₅-b-PPS₇₄-pds, PEG₄₅-b-PPS₄₈-pds, PEG₄₅-b-PPS₂₅-pds) were then purified by double precipitation in cold methanol or diethyl ether. All the polymers were characterized by ¹H NMR (CDCl₃). The dendritic peptide (DP) was conjugated to different PEG-b-PPS polymers via disulfide exchange. PEG-b-PPS (50-100 mg) was reacted with DP (1.2 equiv) in triethylamine/dimethylformamide (DMF) (0.1/1 mL). The peptide-polymer conjugates were purified by repeat precipitation in cold diethyl ether to remove 2-pyridienthione. The vacuum-dried peptide-polymer conjugates were dispersed in water (molecular biology grade) and then dialyzed against water using Slide-A-Lyzer Dialysis Cassettes (20K MWCO, Thermo Fisher Scientific) to remove unreacted peptide. Following purification, the PEG-PPS-ss-DP conjugates were lyophilized.

Preparation of PPDP nanostructures: A variety of PPDP polymers were used in these studies, including: PEG₁₇-b-PPS₈₀-ss-DP (PPDP2), PEG₁₇-b-PPS₅₁-ss-DP (PPDP3), PEG₁₇-b-PPS₄₂-ss-DP (PPDP4), PEG₄₅-b-PPS₇₄-ss-DP (PPDP5), PEG₄₅-b-PPS₄₈-ss-DP (PPDP6), and PEG₄₅-b-PPS₂₅-ss-DP (PPDP7). The specified PPDP polymer was dissolved in water (molecular biology grade) to prepare a stock solution at a 10 mg/mL polymer concentration.

Plasmid DNA-PPDP nanocomplexes (DNA-PPDP) were formed by diluting both PPDPs vectors and pDNA solutions with water to a volume of 50 pL, and were subsequently mixed at a polymer-to-DNA mass ratio (w/w) ranging from 1 to 120. The resulting DNA-PPDP complexes were formed by gentle pipetting for 30 seconds, followed by a 30 minute incubation step at room temperature.

Characterization of PPDP nanostructure morphology and physicochemical properties: The size distribution and zeta potential of the PPDP nanostructures were measured using a Zetasizer Nano instrument (Malvern Instruments). Cryogenic transmission electron microscopy (Cryo-TEM) was performed to characterize nanostructure morphology. Briefly, 200-mesh lacey carbon grids were glow-discharged for 30 seconds in a Pelco easiGlow glow-discharger (Ted Pella Inc.) at 15 mA with a chamber pressure of 0.24 mBar. Grids were prepared with 4 pL of sample and were plunge-frozen into liquid ethane using a FEI Vitrobot Mark III cryo plunge freezing device for 5 seconds with a blot offset of 0.5 mm. After plunge-freezing, grids were loaded into a Gatan 626.5 cryo transfer holder and were imaged at −172° C. in a JEOL JEM1230 LaB6 emission TEM (JEOL USA, Inc.) at 100 kV. Data was acquired using a Gatan Orius 2 k×2 k camera.

Small angle x-ray scattering (SAXS): SAXS was performed at the DuPont-Northwestern-Dow Collaborative Access Team (DND-CAT) beamline at the Advanced Photon Source (APS) at Argonne National Laboratory (Argonne, IL, USA). A ˜7.5 m sample-to-detector distance was used. Silver behenate diffraction patterns were used to calibrate the q-range. Samples were irradiated with 10 keV x-rays using a 3 s exposure time. Data was analyzed in the 0.001-0.5 Å. PRIMUS 2.8.3 and SasView 5.0 software was used for data reduction and model fitting, respectively. Core shell sphere models were fit to the data.

Modeling was performed following established procedures^{[41,47,50]} Characterization of pH-dependent peptide conformational changes: FT-IR spectra were acquired by A Nicolet iS50 FTIR Spectrometer (Thermo Scientific). Spectra were obtained for liquid samples of PPDP2, peptide, and PEG-b-PPS. 64 scans were collected per sample in the 2000-600 cm⁻¹ range. For the circular dichroism (CD) spectroscopy studies, PEG-b-PPS, Peptide and PPDP2 were prepared in water with pH adjusted to 5.5, 6.5, and 7.5 prior to analysis. Samples were prepared in quartz cuvette (0.1 cm path length) and CD spectroscopy was performed using a Jasco J-815 CD Spectrometer. Data was collected via a continuous scan in the 190-300 nm wavelength range using a 100 nm/min scanning speed, a 2 second digital integration time, 2 nm band width, and a 0.5 nm data pitch. The high tension (HT) voltage was monitored to ensure data was collected in the linear range.

Electrophoretic mobility shift assay (EMSA): The stability of PPDP/pDNA nanocomplexes was determined by EMSA. PPDP/pDNA nanocomplexes were prepared at different weight ratios of PPDPs to pDNA, as described elsewhere in this methods section. The same amount of pDNA (0.5 μg) was used for each sample. The obtained nanocomplexes (10 μL) were mixed with loading buffer and loaded on 1% agarose gel containing GelRed® nucleic acid stain submerged in Tris-acetic acid-EDTA (TAE) buffer (40 mM Tris-base, 20 mM acetic acid, 1 mM sodium EDTA). Electrophoresis was carried out at a constant voltage of 100 V for 30 min (Bio-Rad, Inc.). Gels were imaged using a LAS 4010 Gel Imaging system (GE Healthcare).

Cell viability assay: The relative viability of cells transfected with various PPDPs/pDNA, Lipofectamine 2000/pDNA complexes (Lipo2K), PEI/pDNA complexes (PEI with the molecular weight of 25 kDa), and Dendritic peptide (DP1)/pDNA complexes were determined using the 3-(4,5-dimethylthiazol-2-yl)-2,5-diphenyltetrazolium bromide (MTT) assay. Cells were plated in 96-well plates at a seeding density of 30,000 cells per well in 100 μL of culture medium. The cells were then treated under the specified conditions for an incubation period of 24 h (with 0.2 μg of DNA): naked pDNA, Lipofectamine 2000/DNA, PEI (25 kDa)/DNA (w/w from 5:1 to 10:1), DP1/DNA (w/w from 10:1 to 50:1), PPDP2/DNA (w/w from 30:1 to 120:1), PPDP3/DNA (w/w from 30:1 to 120:1), PPDP4/DNA (w/w from 30:1 to 120:1), PPDP5/DNA (w/w from 30:1 to 120:1), PPDP6/DNA (w/w from 30:1 to 120:1), PPDP7/DNA (w/w from 30:1 to 120:1). After 24 h, cells were incubated with the MTT reagent (5 mg/mL in PBS, 10 μL per well) for 4 h. DMSO (200 μL) was used to dissolve the resulting formazan crystals formed in each well. The absorbance of 560 nm light was measured using a SpectraMax M3 multi-mode microplate reader (Molecular Devices, LLC). Cell viability was calculated as the percent viability compared to untreated controls.

Cell transfection in vitro: The pCMV-DsRed (4.6 kb) was used as a small plasmid and pL.CRISPR.EFS.tRFP (11.7 kb) was used as a large plasmid in these studies. RAW 264.7, BMDCs, and Jurkat cells were plated at 105 cells/well and NIH 3T3 fibroblasts were plated at 5×104 cells/well in 24-well plates. For each transfection sample, PPDP-plasmid DNA nanocomplexes (PPDP/DNA) were prepared as follows. Dilute stock solution of PPDP (3 μL of 10 mg/ml PPDP in 50 μL PRMI or DMEM medium without serum) and plasmid DNA stock solution (0.25 μL of 2 mg/ml pDNA in PRMI or DMEM medium without serum). The PPDP/DNA nanocomplexes were prepared by adding 50 μL diluted PPDP suspension into 50 μL diluted pDNA solution. The resulting PPDP/DNA nanocomplexes were formed by gentle pipetting for 30 seconds, and then incubated at room temperature for 30 minutes. Lipofectamine 2000-pDNA complexes were prepared according to the manufacture's instructions. Briefly, dilute plasmid DNA stock solution (0.25 μL of 2 mg/ml pDNA in 50p PRMI or DMEM medium without serum) and mix gently. Dilute Lipofectamine 2000 1 μl in 50 μL PRMI or DMEM medium without serum, and mix gently. After 5 minutes incubation, combine the diluted pDNA with the diluted Lipofectamine 2000, mix gently, and incubate for 20 minutes at room temperature. The 100 μl of naked plasmids, Lipo2K/DNA, PPDP2/DNA, PPDP3/DNA, PPDP4/DNA, PPDP5/DNA, PPDP6/DNA, PPDP7/DNA (w/w 60:1 for PPDP/DNA) suspension was mixed with 400 μl complete medium with serum and added to each well (500 ng plasmid in 500 μl medium per well). After a 48 h transfection period, the transfection efficiency (percentages of DsRed+ and RFP+ cells) and the mean fluorescence intensity (MFI) were quantified by flow cytometry using a BD LSRFortessa 6-Laser flow cytometer (BD Biosciences). FlowJo software was used to analyze the acquired flow cytometry data. For confocal microscopy analysis, RAW 264.7 and NIH 3T3 cells were plated at 104 cells/well in 8-well Chamber slides (Thermo Fisher Scientific) and were cultured for 24 h before use. Cells were then transfected with a naked plasmid, Lipo2K/DNA, and PPDP2/DNA (w/w 60:1 for PPDP2/DNA), respectively, with 500 ng plasmid per well. After 48 h, cells were counterstained with NucBlue™ Live ReadyProbes™ Reagent (nuclei stain, one drop) for 15 min in the dark. Images were acquired on a Leica TCS SP8 confocal microscope with a 40× oil immersion objective.

Cellular internalization analysis: Small plasmid DNA (pcDNA3.1, 5.4 kb) was fluorescently labeled with Alexa Fluor 488™ Ulysis™ Nucleic Acid Labelling Kit (Thermo Fisher Scientific) using manufacturer procedures. RAW 264.7 cells were prepared at 20,000 cells per well in 8-well Chamber slides (Thermo Fisher Scientific) and were cultured for 24 h before use. Alexa Fluor 488-labeled pDNA (488-DNA) was mixed with PPDP2 (w/w 60:1 for PPDP2: plasmid), or Lipo2K (v/w 6 μl/g for Lipo2K: plasmid) as described above. The obtained 488-pDNA PPDP2 complexes (488-pDNA-PPDP2), 488-pDNA, and 488-pDNA-Lipo2K were incubated with cells for 1 h, 4 h, or 18 h incubation periods, as specified. The concentration of 488-pDNA was 1 μg/mL for each well. After incubation, cells were washed twice with PBS and were subsequently incubated with LysoTracker™ Red DND-99 (1:5000 dilution, 300 μL DMEM) for 30 min. Afterwards, the cells were washed twice with PBS, and were incubated with NucBlue™ Live ReadyProbes™ Reagent (nuclei stain, 1 drop) in 300 μL PBS per well for 15 min in the dark. Images were acquired on a Leica TCS SP8 confocal microscope with a 63× oil immersion objective.

Statistical Analysis: GraphPad Prism software (version 8) was used for data analysis. Data are presented as mean±SD. Significance was determined using an appropriate statistical test, as described in the corresponding figure legends.

### REFERENCES FOR EXAMPLE 1

### References

[1] R. S. Riley, C. H. June, R. Langer, M. J. Mitchell, Nat Rev Drug Discov 2019, 18, 175.
[2] R. A. Larocca, P. Abbink, J. P. S. Peron, P. M. de A. Zanotto, M. J. Iampietro, A. Badamchi-Zadeh, M. Boyd, D. Ng'ang'a, M. Kirilova, R. Nityanandam, N. B. Mercado, Z. Li, E. T. Moseley, C. A. Bricault, E. N. Borducchi, P. B. Giglio, D. Jetton, G. Neubauer, J. P. Nkolola, L. F. Maxfield, R. A. De La Barrera, R. G. Jarman, K. H. Eckels, N. L. Michael, S. J. Thomas, D. H. Barouch, Nature 2016, 536, 474.
[3] T. R. F. Smith, A. Patel, S. Ramos, D. Elwood, X. Zhu, J. Yan, E. N. Gary, S. N. Walker, K. Schultheis, M. Purwar, Z. Xu, J. Walters, P. Bhojnagarwala, M. Yang, N. Chokkalingam, P. Pezzoli, E. Parzych, E. L. Reuschel, A. Doan, N. Tursi, M. Vasquez, J. Choi, E. Tello-Ruiz, I. Maricic, M. A. Bah, Y. Wu, D. Amante, D. H. Park, Y. Dia, A. R. Ali, F. I. Zaidi, A. Generotti, K. Y. Kim, T. A. Herring, S. Reeder, V. M. Andrade, K. Buttigieg, G. Zhao, J.-M. Wu, D. Li, L. Bao, J. Liu, W. Deng, C. Qin, A. S. Brown, M. Khoshnejad, N. Wang, J. Chu, D. Wrapp, J. S. McLellan, K. Muthumani, B. Wang, M. W. Carroll, J. J. Kim, J. Boyer, D. W. Kulp, L. M. P. F. Humeau, D. B. Weiner, K. E. Broderick, Nat Commun 2020, 11, 2601.
[4] S. Chang, E. L. Mahon, H. A. MacKay, W. H. Rottmann, S. H. Strauss, P. M. Pijut, W. A. Powell, V. Coffey, H. Lu, S. D. Mansfield, T. J. Jones, In Vitro Cell. Dev. Biol.-Plant 2018, 54, 341.
[5] C. E. Dunbar, K. A. High, J. K. Joung, D. B. Kohn, K. Ozawa, M. Sadelain, Science n.d., 359, DOI 10.1126/science.aan4672.
[6] T. I. Cornu, C. Mussolino, T. Cathomen, Nat Med 2017, 23, 415.
[7] H. Li, Y. Yang, W. Hong, M. Huang, M. Wu, X. Zhao, Signal Transduction and Targeted Therapy 2020, 5, 1.
[8] G. J. Knott, J. A. Doudna, Science 2018, 361, 866.
[9] L. Cong, F. Zhang, Methods Mol Biol 2015, 1239, 197.
[10] C. Liu, L. Zhang, H. Liu, K. Cheng, J Control Release 2017, 266, 17.
[11] B. H. Yip, Biomolecules 2020, 10, DOI 10.3390/biom10060839.
[12] H. Song, M. Yu, Y. Lu, Z. Gu, Y. Yang, M. Zhang, J. Fu, C. Yu, J. Am. Chem. Soc. 2017, 139, 18247.
[13] B. Shi, M. Zheng, W. Tao, R. Chung, D. Jin, D. Ghaffari, O. C. Farokhzad, Biomacromolecules 2017, 18, 2231.
[14] R. M. David, A. T. Doherty, Toxicological Sciences 2017, 155, 315.
[15] A. Baldo, E. van den Akker, H. E. Bergmans, F. Lim, K. Pauwels, Curr Gene Ther 2013, 13, 385.
[16] J. L. Shirley, Y. P. de Jong, C. Terhorst, R. W. Herzog, Molecular Therapy 2020, 28, 709.
[17] N. F. Nidetz, M. C. McGee, L. V. Tse, C. Li, L. Cong, Y. Li, W. Huang, Pharmacology & Therapeutics 2020, 207, 107453.
[18] R. Goswami, G. Subramanian, L. Silayeva, I. Newkirk, D. Doctor, K. Chawla, S. Chattopadhyay, D. Chandra, N. Chilukuri, V. Betapudi, Front. Oncol. 2019, 9, DOI 10.3389/fonc.2019.00297.
[19] N. Nayerossadat, T. Maedeh, P. A. Ali, Adv Biomed Res 2012, 1, DOI 10.4103/2277-9175.98152.
[20] E. Ayuso, Mol Ther Methods Clin Dev 2016, 3, 15049.
[21] M. G. Moleirinho, R. J. S. Silva, P. M. Alves, M. J. T. Carrondo, C. Peixoto, Expert Opin Biol Ther 2020, 20, 451.
[22] A. Srivastava, K. M. G. Mallela, N. Deorkar, G. Brophy, J Pharm Sci 2021, DOI 10.1016/j.xphs.2021.03.024.
[23] J. C. M. van der Loo, J. F. Wright, Hum Mol Genet 2016, 25, R42.
[24] Y. Zhang, A. Satterlee, L. Huang, Mol Ther 2012, 20, 1298.
[25] M. Ramamoorth, A. Narvekar, J Clin Diagn Res 2015, 9, GE01.
[26] A. B. Hil, M. Chen, C.-K. Chen, B. A. Pfeifer, C. H. Jones, Trends Biotechnol 2016, 34, 91.
[27] M. P. Stewart, A. Sharei, X. Ding, G. Sahay, R. Langer, K. F. Jensen, Nature 2016, 538, 183.
[28] O. Boussif, F. Lezoualc'h, M. A. Zanta, M. D. Mergny, D. Scherman, B. Demeneix, J. P. Behr, Proc Natl Acad Sci USA 1995, 92, 7297.
[29] D. Fischer, T. Bieber, Y. Li, H. P. Elsasser, T. Kissel, Pharm Res 1999, 16, 1273.
[30] W. T. Godbey, K. K. Wu, A. G. Mikos, J Biomed Mater Res 1999, 45, 268.
[31] X. Xu, Y. Jian, Y. Li, X. Zhang, Z. Tu, Z. Gu, ACS Nano 2014, 8, 9255.
[32] H. Zeng, M. E. Johnson, N. J. Oldenhuis, T. N. Tiambeng, Z. Guan, ACS Cent. Sci. 2015, 1, 303.
[33] D. Zhou, L. Cutlar, Y. Gao, W. Wang, J. O'Keeffe-Ahern, S. McMahon, B. Duarte, F. Larcher, B. J. Rodriguez, U. Greiser, W. Wang, Science Advances 2016, 2, e1600102.
[34] S. Wang, R. Chen, Chem Mater 2017, 29, 5806.
[35] N. Malik, R. Wiwattanapatapee, R. Klopsch, K. Lorenz, H. Frey, J. W. Weener, E. W. Meijer, W. Paulus, R. Duncan, J Control Release 2000, 65, 133.
[36] A. Kwok, G. A. Eggimann, J.-L. Reymond, T. Darbre, F. Hollfelder, ACS Nano 2013, 7, 4668.
[37] E. Abbasi, S. F. Aval, A. Akbarzadeh, M. Milani, H. T. Nasrabadi, S. W. Joo, Y. Hanifehpour, K. Nejati-Koshki, R. Pashaei-Asl, Nanoscale Res Lett 2014, 9, 247.
[38] K. Madaan, S. Kumar, N. Poonia, V. Lather, D. Pandita, J Pharm Bioallied Sci 2014, 6, 139.
[39] S. Yi, S. D. Allen, Y.-G. Liu, B. Z. Ouyang, X. Li, P. Augsornworawat, E. B. Thorp, E. A. Scott, ACS Nano 2016, 10, 11290.
[40] S. Yi, X. Zhang, M. H. Sangji, Y. Liu, S. D. Allen, B. Xiao, S. Bobbala, C. L. Braverman, L. Cai, P. I. Hecker, M. DeBerge, E. B. Thorp, R. E. Temel, S. I. Stupp, E. A. Scott, Adv Funct Mater 2019, 29, 1904399.
[41] M. P. Vincent, S. Bobbala, N. B. Karabin, M. Frey, Y. Liu, J. O. Navidzadeh, T. Stack, E. A. Scott, Nat Commun 2021, 12, 648.
[42] A. E. Vasdekis, E. A. Scott, C. P. O'Neil, D. Psaltis, Jeffrey. A. Hubbell, ACS Nano 2012, 6, 7850.
[43] E. A. Scott, A. Stano, M. Gillard, A. C. Maio-Liu, M. A. Swartz, J. A. Hubbell, Biomaterials 2012, 33, 6211.
[44] S. Allen, O. Osorio, Y.-G. Liu, E. Scott, J Control Release 2017, 262, 91.
[45] S. Allen, M. Vincent, E. Scott, J Vis Exp 2018, e57793.
[46] S. Bobbala, S. D. Allen, S. Yi, M. Vincent, M. Frey, N. B. Karabin, E. A. Scott, Nanoscale 2020, 12, 5332.
[47] T. Stack, M. Vincent, A. Vahabikashi, G. Li, K. M. Perkumas, W. D. Stamner, M. Johnson, E. Scott, Small 2020, 2004205.
[48] T. Stack, Y. Liu, M. Frey, S. Bobbala, M. Vincent, E. Scott, Nanoscale Horiz. 2021, 6, 393.
[49] M. P. Vincent, T. Stack, A. Vahabikashi, G. Li, K. M. Perkumas, R. Ren, H. Gong, W. D. Stamner, M. Johnson, E. A. Scott, bioRxiv 2021, 2021.05.19.444878.
[50] M. P. Vincent, N. B. Karabin, S. D. Allen, B. Sharan, M. A. Frey, S. Yi, Y. Yang, E. A. Scott, Adv Ther 2021, 2100062, DOI 10.1002/adtp.202100062.
[51] S. D. Allen, Y.-G. Liu, S. Bobbala, L. Cai, P. I. Hecker, R. Temel, E. A. Scott, Nano Res. 2018, 11, 5689.
[52] D. J. Dowling, E. A. Scott, A. Scheid, I. Bergelson, S. Joshi, C. Pietrasanta, S. Brightman, G. Sanchez-Schmitz, S. D. Van Haren, J. Ninkovid, D. Kats, C. Guiducci, A. de Titta, D. K. Bonner, S. Hirosue, M. A. Swartz, J. A. Hubbell, O. Levy, Journal of Allergy and Clinical Immunology 2017, 140, 1339.
[53] J. D. Perlmutter, M. F. Hagan, Annu Rev Phys Chem 2015, 66, 217.
[54] R. D. Requiao, R. L. Cameiro, M. H. Moreira, M. Ribeiro-Alves, S. Rossetto, F. L. Palhano, T. Domitrovic, Scientific Reports 2020, 10, 5470.
[55] M. G. Rossmann, Q Rev Biophys 2013, 46, 133.
[56] J. E. Summerton, Ann N Y Acad Sci 2005, 1058, 62.
[57] S. W. Provencher, J. Glockner, Biochemistry 1981, 20, 33.
[58] V. N. Uversky, J. R. Gillespie, A. L. Fink, Proteins: Structure, Function, and Genetics 2000, 41, 415.
[59] A. K. Dunker, J. D. Lawson, C. J. Brown, R. M. Williams, P. Romero, J. S. Oh, C. J. Oldfield, A. M. Campen, C. M. Ratliff, K. W. Hipps, J. Ausio, M. S. Nissen, R. Reeves, C. Kang, C. R. Kissinger, R. W. Bailey, M. D. Griswold, W. Chiu, E. C. Garner, Z. Obradovic, Journal of Molecular Graphics and Modelling 2001, 19, 26.
[60] R. Linding, J. Schymkowitz, F. Rousseau, F. Diella, L. Serrano, J Mol Biol 2004, 342, 345.
[61] V. N. Uversky, C. J. Oldfield, A. K. Dunker, Journal of Molecular Recognition 2005, 18, 343.
[62] M. E. Oates, P. Romero, T. Ishida, M. Ghalwash, M. J. Mizianty, B. Xue, Z. Dosztinyi, V. N. Uversky, Z. Obradovic, L. Kurgan, A. K. Dunker, J. Gough, Nucleic Acids Research 2012, 41, D508.
[63] M. Vincent, S. Schnell, Scientific Data 2016, 3, 160045.
[64] M. Vincent, M. Whidden, S. Schnell, Biophysical Chemistry 2016, 213, 6.
[65] M. Vincent, S. Schnell, 2017, DOI 10.1101/060699.
[66] M. Vincent, V. N. Uversky, S. Schnell, Proteomics 2019, 19, e1800415.
[67] B. Wang, S. A. Merillat, M. Vincent, A. K. Huber, V. Basrur, D. Mangelberger, L. Zeng, K. Elenitoba-Johnson, R. A. Miller, D. N. Irani, A. A. Dlugosz, S. Schnell, K. M.
Scaglione, H. L. Paulson, Journal of Biological Chemistry 2016, 291, 3030.
[68] P. Kumar, M. Bansal, J Struct Biol 2016, 196, 414.
[69] J. L. S. Lopes, A. J. Miles, L. Whitmore, B. A. Wallace, Protein Science 2014, 23, 1765.
[70] M. Lella, R. Mahalakshmi, J Pept Sci 2017, 108, DOI https://doi.org/10.1002/bip.22894.
[71] A. L. Rucker, T. P. Creamer, Protein Sci 2002, 11, 980.
[72] M. L. Tiffany, S. Krimm, Biopolymers 1968, 6, 1767.
[73] M. L. Tiffany, S. Krimm, Biopolymers 1972, 11, 2309.
[74] A. I. Arunkumar, T. K. Kumar, C. Yu, Biochim Biophys Acta 1997, 1338, 69.
[75] A. Erazo-Oliveras, N. Muthukrishnan, R. Baker, T.-Y. Wang, J.-P. Pellois, Pharmaceuticals (Basel) 2012, 5, 1177.
[76] N. Ohmori, T. Niidome, A. Wada, T. Hirayama, T. Hatakeyama, H. Aoyagi, Biochem Biophys Res Commun 1997, 235, 726.
[77] A. El-Sayed, S. Futaki, H. Harashima, AAPS J2009, 11, 13.
[78] A. Napoli, N. Tirelli, G. Kilcher, A. Hubbell, Macromolecules 2001, 34, 8913.
[79] S. Cerritelli, D. Velluto, J. A. Hubbell, Biomacromolecules 2007, 8, 1966.
[80] S. Cerritelli, C. P. O'Neil, D. Velluto, A. Fontana, M. Adrian, J. Dubochet, J. A. Hubbell, Langmuir 2009, 25, 11328.

We claim:

1. A synthetic PEG-b-PPS-linker-DP polymer for producing nanostructures comprising a poly(ethylene glycol)-block-poly(propylene sulfide) copolymer (PEG-b-PPS) conjugated with a dendritic-specific branched cationic peptide (DP).

2. The synthetic PEG-b-PPS-linker-DP polymer of claim 1, wherein the PEG-b-PPS is conjugated via linker, optionally wherein the linker is a disulfide bond (-ss-).

3. The synthetic PEG-b-PPS-linker-DP polymer of claim 1, wherein (a) the PEG-b-PPS has a PEG weight fraction of 0.17-0.45; (b) the PEG-b-PPS has a PPS weight fraction of 0.25-0.80, or
(c) both (a) and (b).

4. The synthetic PEG-b-PPS-linker-DP polymer of any one of the preceding claims, wherein the DP is a peptide of SEQ ID NO: 1.

5. The synthetic PEG-b-PPS-linker-DP polymer of any one of the preceding claims, wherein the polymer is PEGₘ-b-PPSₙ, wherein m and n are integers each selected from 1-500.

6. A nanocarrier system for delivering nucleic acids to a cell, the system comprising:
(a) a nanostructure comprising poly(ethylene glycol)-block-poly(propylene sulfide) copolymer (PEG-b-PPS) conjugated with a dendritic-specific branched cationic peptide (DP) of any one of claims 1-5; and
(b) a nucleic acid selected from the group consisting of DNA and RNA.

7. The nanocarrier system of claim 6, wherein the nanostructure of PEG-b-PPS-linker-DP has a PEG weight fraction of 0.17-0.45.

8. The nanocarrier system of claim 6 or 7, wherein the nanostructure of PEG-b-PPS-linker-DP has a PPS weight fraction of 0.25-0.80.

9. The nanocarrier system of any one of claims 6-8, wherein the DP of the nanostructure of PEG-b-PPS-linker-DP is a peptide of SEQ ID NO: 1.

10. The nanocarrier system of any one of claims 6-9, wherein the polynucleotide is DNA, preferably wherein the DNA is a plasmid DNA, a DNA construct, or a polynucleotide sequence encoding a protein, peptide or fragment thereof of interest.

11. The nanocarrier system of claim 10, wherein the mass ratio (w/w) of PEG-b-PPS-ss-DP:DNA is 5:1 to 50:1.

12. The nanocarrier system of claim 10, wherein the mass ratio (w/w) of PEG-b-PPS-ss-DP:DNA is 15:1 to 120:1.

13. The nanocarrier system of any one of claims 6-12, wherein the polymer component is PEGₘ-b-PPSₙ, wherein m and n are each integers selected from 1-500.

14. A method of delivering a polynucleotide sequence to a cell, the method comprising contacting or administering to the cell the nanocarrier system of any one of claims 6-13.

15. The method of claim 14, wherein the DP of the nanostructure of PEG-b-PPS-linker-DP is a peptide of SEQ ID NO: 1.

16. The method of claim 14 or 15, wherein the polynucleotide is DNA or RNA.

17. The method of any one of claims 14-16, wherein the mass ratio (w/w) of PEG-b-PPS-linker-DP:DNA is 5:1 to 50:1.

18. The method of any one of claims 14-17, wherein the mass ratio (w/w) of PEG-b-PPS-linker-DP:DNA is 15:1 to 120:1.

19. The method of any one of claims 14-18, wherein the conjugated DP does not result in cytotoxicity to the cell compared to the unconjugated DP.

20. The method of any one of claims 14-19, wherein the cell is an immune cell, preferably a dendritic cell.

21. The method of any one of claims 14-20, wherein the cell is in vitro.

22. A method of transfecting an immune cell to deliver a polynucleotide sequence to the nucleus of the immune cell, the method comprising contacting the immune cell with the system of any one of claims 6-13 for a sufficient time to deliver the polynucleotide sequence to the nucleus of the immune cell.

23. The method of claim 22, wherein the immune cell is in vitro.

24. The method of claim 21 or 22, wherein the polynucleotide encodes a therapeutic agent or cytokine.

25. A non-toxic method of transducing a cell, the method comprising:
a) contacting the cell in culture with the nanocarrier system of any one of claims 6-12, and
b) culturing the cell for a sufficient time to allow the polynucleotide to be delivered to the cell, wherein the nanocarrier is non-toxic to the cell.

26. The method of claim 25, wherein step (a) and (b) the cell is cultured in medium comprising serum.

27. The method of claim 25 or 26, wherein the method is in vitro.

28. The method of any one of claims 25-27, wherein at least 50% of the cells are transduced with the polynucleotide.

29. The method of any one of claims 25-28, wherein the cell is an immune cell, preferably a dendritic cell.
